(19) United States
(12) Patent Application Publication
(10) Pub. No.: US 2021/0346634 A1
(43) Pub. Date: Nov. 11, 2021

(54) METHODS AND APPARATUS FOR CONTROLLING RESPIRATORY THERAPY WITH SUPPLEMENTARY OXYGEN

(71) Applicant: ResMed Pty Ltd, Bella Vista, NSW (AU)

(72) Inventors: Dion Charles Chewe MARTIN, Sydney (AU); Paul Andrew DICKENS, Sydney (AU)

(73) Assignee: ResMed Pty Ltd, Bella Vista, NSW (AU)

(21) Appl. No.: 17/270,643

(22) PCT Filed: Aug. 23, 2019
(86) PCT No.: PCT/AU2019/050892

§ 371 Date: Feb. 23, 2021

(30) Foreign Application Priority Data
Aug. 23, 2018 (AU) 2018903114

(51) Int. Cl. A61M 16/10; A61M 16/00; A61M 16/06; A61M 16/16; A61M 16/20

(52) U.S. Cl. CPC A61M 16/101; A61M 16/024; A61M 16/0672; A61M 16/16; A61M 16/202; A61M 16/208; A61M 2016/0027; A61M 2016/1025; A61M 2205/3375; A61M 2205/3334; A61M 2205/3553; A61M 2202/0208; A61M 2016/003

(57) ABSTRACT

Apparatus and methods provide operations for a respiratory disorder therapy by generating a flow or pressure therapy with supplementary oxygen. The method may be implemented by one or more processors that may set one or more therapy parameters associated with generating a flow of air to a patient interface via an air circuit. The one or more processors may set one or more supplementary oxygen parameters associated with introducing supplementary oxygen into the flow of air at the air circuit. The method may involve computing an oxygen performance metric associated with characteristic(s) of a patient and the air flow with the supplementary oxygen. The computing may include applying function(s) comprising the therapy parameter(s) and supplementary oxygen parameter(s). Output, such as a display indicator or automated therapy control change(s), may be generated using the computed oxygen performance metric. Changes to therapy parameter(s) may be made to optimise the metric.

### 1 CROSS-REFERENCE TO RELATED APPLICATIONS

This application claims the benefit of Australian Provisional Application No. 2018903114, filed 23 Aug. 2018, the entire disclosures of which are hereby incorporated herein by reference.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use, such as those involving an oxygen source when implemented for use with other respiratory pressure or flow therapy devices.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, ninth edition, published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), invasive ventilation (IV), and high flow therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients who are no longer able to effectively breathe by themselves, and may be provided using a tracheostomy tube. In some forms, the comfort and effectiveness of these therapies may be improved.

### 2.2.2.2 Respiratory Flow Therapies

Not all respiratory therapies aim to deliver a prescribed therapy pressure. Some respiratory therapies aim to deliver a prescribed respiratory volume, possibly by targeting a flow rate profile over a targeted duration. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, COPD and other respiratory disorders. One mechanism of action is that the high flow rate of air at the airway entrance improves ventilation efficiency by flushing, or washing out, expired CO₂ from the patient's anatomical deadspace. HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched gas at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 2.2.3 Respiratory Therapy Systems

These respiratory therapies may be provided by a respiratory therapy system or device. A respiratory therapy system may comprise a Respiratory Therapy Device (RPT device), a patient interface, an air circuit, a humidifier, and an oxygen source.

### 2.2.3.1 Respiratory Therapy (RPT) Device

A respiratory therapy (RPT) device is configured to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include CPAP devices and ventilators.

### 2.2.3.2 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. For pressure therapies, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For flow therapies such as nasal HFT, the patient interface may be configured to insufflate the nares but specifically to avoid a complete seal. One example of such an unsealed patient interface is a nasal cannula.

### 2.2.3.3 Air Circuit

An air circuit is a conduit or a tube constructed and arranged to allow, in use, a flow of air to travel between two components of a therapy system such as the RPT device and the patient interface. In some cases, there may be separate limbs of the air circuit for inhalation and exhalation. In other cases, a single limb air circuit is used.

### 2.2.3.4 Humidifier

Delivery of a flow of air without humidification may cause drying of the airways. The use of a humidifier with an RPT device produces humidified air that minimizes drying of the nasal mucosa and increases patient airway comfort. In addition, in cooler climates, warm air applied generally to the face area in and about the patient interface is more comfortable than cold air. Humidifiers therefore often have the capacity to heat the flow of air as well as humidifying it.

### 2.2.3.5 Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption. This may be achieved by delivering the oxygen as series of pulses or “boli”, each bolus timed to coincide with the start of inspiration. This therapy mode is known as pulsed or demand (oxygen) delivery (POD) mode, in contrast with traditional continuous flow delivery more suited to stationary oxygen concentrators. POD mode, while avoiding the waste of delivering oxygen during expiration, still has the potential to waste oxygen, and thereby impede efficiency, in at least two ways:
Any portion of the bolus whose flow rate exceeds the instantaneous inspiratory flow rate may not be inspired during the current breath. For example, some of this portion may flow back out of the patient's nostrils (retrograde flow) to atmosphere (retrograde flow waste).
Due to the nature of mammalian respiration—that of tidal breathing via conduit airways to an internal lung—not all of the inspiratory flow reaches the gas-exchanging areas of the lung; the end portion of each inspiration remains in the conduit airways (the anatomical deadspace) and is exhaled without reaching the alveoli. Therefore, oxygen delivered during the latter part of inspiration will only reach the anatomical deadspace (deadspace waste).

Oxygen for supplementary oxygen therapy may be delivered to one or more points in the pneumatic path of the main respiratory therapy, such as within the RPT device, within the air circuit, or directly to the patient interface. Key performance metrics for supplementary oxygen therapy are the fraction of oxygen at the entrance to the patient's lung (the FiO₂), the oxygen supplementation ratio, which is the ratio of the volume of supplementary oxygen entering the lung (reaching the alveoli) per breath to the inspiratory volume, and the oxygen delivery efficiency, which is the volume ratio of supplementary oxygen to supplementary oxygen delivered into the system, calculated per breath. Maximising the oxygen delivery efficiency is equivalent to minimising oxygen waste.

POCs operating in POD mode traditionally do not function efficiently when coupled to the airpath of RPT devices distally to the patient interface, for at least the following reasons:
the positive pressure within the device's air circuit confounds the POC's triggering scheme (which is typically based on sensing negative pressure in the conduit).
Even if triggering is successfully achieved, the bolus of oxygen may not be received in time to reach the alveoli, e.g. due to the propagation delay along the portion of the air circuit that must be traversed by the bolus (a portion referred to as the oxygen circuit).

Even when a POC is operating in continuous flow mode when coupled to the airpath of an RPT device delivering respiratory pressure therapy, varying device flow rates over the respiratory cycle may lead to oxygen accumulation and “POD-like” behaviour. Consequently, oxygen delivery efficiency may be impaired in this scenario as well.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficiency, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

One form of the present technology comprises methods and apparatus for triggering a POD-mode POC that is ignorant of the respiratory therapy device to which it is supplying supplementary oxygen. The apparatus comprises a negative-pressure-inducing module between the POC and the respiratory therapy device, controllable by the respiratory therapy device controller to deliver the negative pressure needed to trigger the POC while not affecting the POC at other times.

Another form of the present technology comprises methods and apparatus for estimating an oxygen performance metric such as the fraction of inspired oxygen (FiO₂) or oxygen supplementation ratio when a supplementary oxygen source is delivering supplementary oxygen into the air circuit of a respiratory therapy device. The method comprises using an air circuit model and parameters of the therapy device, air circuit, oxygen source, and patient to estimate the oxygen performance metric. Optionally, the method may implement or recommend changes to the parameters to improve the oxygen performance metric.

Some forms of the present technology may include a method of one or more processors for operation of apparatus configured to generate a respiratory therapy with supplementary oxygen for a respiratory disorder of a patient. The method may include setting, by the one or more processors, one or more therapy parameters associated with delivering a flow of air to a patient interface via an air circuit of the apparatus. The method may include setting, by the one or more processors, one or more supplementary oxygen parameters associated with inserting supplementary oxygen into the flow of air of the air circuit. The method may include computing, by the one or more processors, an oxygen performance metric associated with (1) one or more characteristics of the patient and (2) the flow of air with the supplementary oxygen, the computing including applying one or more functions may include the one or more therapy parameters and the one or more supplementary oxygen parameters. The method may include generating, by the one or more processors, output based on the computed oxygen performance metric.

In some versions, the output may include a displayed indicator including the computed oxygen performance metric. The output may include an adjustment of one or more of: the one or more therapy parameters; and the one or more supplementary oxygen parameters, for improving the oxygen performance metric. The adjustment may include an automatic change to a setting of one or more controllers of the apparatus by the one or more processors. The one or more functions may include one or more parameters of the air circuit. The one or more functions may include a difference in a volume of oxygen entering the patient's lung and a volume of oxygen expected to enter the lung in an absence of therapy. The one or more functions may include a ratio of the difference and an inspiratory volume of the patient. The one or more functions may include a ratio of the difference and a bolus volume of the supplementary oxygen. The oxygen performance metric may be oxygen delivery efficiency.

In some versions, computing the oxygen performance metric may include determining a ratio of (a) a difference in a volume of oxygen entering the patient's lung minus a volume of oxygen expected to enter the lung in an absence of therapy, and (b) a bolus volume of the supplementary oxygen. The computing may include applying a pipe transport model of the air circuit. The applying may include generating a mole fraction vector of gas mixture at an entrance to the patient's lungs. The computing the oxygen performance metric may include accessing data may include one or more signals representing measurement of properties of the flow of air using one or more sensors, the properties corresponding with either or both of (a) the one or more therapy parameters; and (b) the one or more supplementary oxygen parameters. The one or more therapy parameters may include one or more of a device flow rate and a vent flow rate. The one or more supplementary oxygen parameters may include a supplementary oxygen flow rate. The one or more characteristics of the patient may include a respiratory flow rate.

In some versions, the computing may include approximating a respiratory flow rate profile of the patient by fitting a model flow rate profile to breathing parameters of the patient. The breathing parameters may include one or more of tidal volume, breathing rate, and duty cycle. The respiratory therapy may be respiratory pressure therapy, and the one or more therapy parameters may include a treatment pressure. Inserting supplementary oxygen may include operating an oxygen source in a pulsed oxygen delivery mode, wherein release of the supplementary oxygen may be controlled with a bolus advance, a bolus duration, and a bolus flow rate.

In some versions, the method may include generating a pseudo-trigger signal. The pseudo-trigger signal may be configured to activate a pneumatic intermediary module to generate a pressure drop for triggering of the release of the supplementary oxygen as a bolus. The computing may include estimating a vent flow rate of the patient using signals representing flow rate and pressure respectively of the flow of air and one or more parameters of the air circuit. The computing may include estimating a respiratory flow rate of the patient using the signals representing flow rate and pressure respectively of the flow of air. The computing may include: computing the oxygen performance metric using the vent flow rate, the respiratory flow rate, the signal representing flow rate, a supplementary oxygen flow rate, and one or more parameters of the air circuit. The one or more parameters of the air circuit may include: an insertion point of the supplementary oxygen; and a volume of the air circuit.

Some forms of the present technology may include a respiratory therapy system with supplementary oxygen. The system may include a respiratory therapy device configured to generate a flow of air and adapted to pneumatically couple with (a) a patient interface configured to deliver the flow of air to an entrance to an airway of a patient, and (b) an air circuit configured to conduct the flow of air between the respiratory therapy device and the patient interface. The system may include an oxygen source configured to insert supplementary oxygen into the flow of air. The system may include a controller. The controller may be configured to control setting of the respiratory therapy device to generate a respiratory therapy to the patient according to one or more therapy parameters. The controller may be configured to control setting of the oxygen source to insert the supplementary oxygen into the flow of air according to one or more supplementary oxygen parameters. The controller may be configured to compute an oxygen performance metric associated with (1) one or more characteristics of the patient and (2) the flow of air with the supplementary oxygen, the computing including applying one or more functions may include the one or more therapy parameters and the one or more supplementary oxygen parameters. The controller may be configured to generate output based on the computed oxygen performance metric.

In some versions, the output may include a displayed indicator including the computed oxygen performance metric. The output may include an adjustment of one or more of the one or more therapy parameters, and the one or more supplementary oxygen parameters, for improving the oxygen performance metric. The controller may be a controller of the respiratory therapy device.

Some forms of the present technology may include a respiratory therapy system for generating a respiratory therapy with supplementary oxygen for a respiratory disorder. The system may include a respiratory therapy device configured to generate a flow of air and adapted to pneumatically couple with (a) a patient interface configured to deliver the flow of air to an entrance to an airway of a patient, and (b) an air circuit configured to conduct the flow of air between the respiratory therapy device and the patient interface. The system may include an oxygen source configured to insert supplementary oxygen into the flow of air. The system may include an a controller. The controller may be configured with processor executable instructions. The processor executable instructions may be configured to control operation of the system to generate the therapy. The processor executable instructions may include instructions to perform any one or more or all of the aspects of the methods described herein.

Some forms of the present technology may include a processor-readable medium, having stored thereon processor-executable instructions which, when executed by one or more processors of one or more controllers, cause the one or more controllers to control operation of apparatus configured to generate a therapy with supplementary oxygen for a respiratory disorder of a patient according to any one or more or all of the aspects of the methods described herein.

Some forms of the present technology may include apparatus. The apparatus may include means for generating a flow of air. The apparatus may include means for delivering the flow of air to an entrance to an airway of a patient. The apparatus may include means for conducting the flow of air between the means for generating and the means for delivering. The apparatus may include means for inserting supplementary oxygen into the flow of air. The apparatus may include means for controlling the means for generating to deliver a respiratory therapy to the patient according to one or more therapy parameters. The apparatus may include means for controlling the means for delivering to insert the supplementary oxygen into the flow of air according to one or more supplementary oxygen parameters. The apparatus may include means for computing an oxygen performance metric associated with (1) one or more characteristics of the patient and (2) the flow of air with the supplementary oxygen. The computing may include applying one or more functions that may include the one or more therapy parameters and the one or more supplementary oxygen parameters. The apparatus may include means for generating output based on the computed oxygen performance metric.

Some forms of the present technology may include a method of one or more processors for optimising one or more parameters of a respiratory therapy system with supplementary oxygen. The method of the one or more processors may include optimising the one or more parameters of the respiratory therapy system with respect to an oxygen performance metric of the respiratory therapy system, to obtain optimal values of the one or more parameters. The method of the one or more processors may include setting automatically controllable ones of the one or more parameters of the respiratory therapy system to the respective optimal values of the automatically controllable ones of the one or more parameters. The method of the one or more processors may include generating a recommendation to a user, via an interface of the respiratory therapy system, with the optimal values of manually controllable ones of the one or more parameters of the respiratory therapy system.

In some versions, the optimising may include optimising, for each combination of discretely controllable ones of the one or more parameters, continuously controllable ones of the one or more parameters, giving the optimal values of the continuously controllable parameters for a current combination of discretely controllable parameters. The optimising may include selecting the combination of discretely controllable parameters for which the optimal values of the continuously controllable parameters give the highest oxygen performance metric, together with corresponding optimal values of the continuously controllable parameters. The optimising the continuously controllable parameters may include, by the one or more processors, estimating the oxygen performance metric for current values of the continuously controllable parameters. The optimising the continuously controllable parameters may include, by the one or more processors, adjusting values of the continuously controllable parameters so as to improve the oxygen performance metric. The optimising the continuously controllable parameters may include, by the one or more processors, repeating the estimating and adjusting until the estimated oxygen performance metric satisfies a threshold.

In some versions, estimating the oxygen performance metric may include estimating a respiratory flow rate of a patient based on a height of the patient. Estimating the oxygen performance metric may include estimating a vent flow rate of the respiratory therapy system using one or more parameters of the respiratory therapy and the estimated respiratory flow rate. Estimating the oxygen performance metric may include estimating the oxygen performance metric using one or more parameters of an air circuit of the respiratory therapy system, the estimated vent flow rate, a respiratory therapy device flow rate, a supplementary oxygen flow rate, and the estimated respiratory flow rate.

Some forms of the present technology may include a respiratory therapy system with supplementary oxygen. The system may include a respiratory therapy device configured to generate a flow of air and adapted to pneumatically couple with (a) a patient interface configured to deliver the flow of air to an entrance to an airway of a patient, and (b) an air circuit configured to conduct the flow of air between the respiratory therapy device and the patient interface. The system may include an oxygen concentrator configured to insert supplementary oxygen into the flow of air. The system may include a controller. The controller may be configured to optimise one or more parameters of the respiratory therapy system with respect to an oxygen performance metric of the respiratory therapy system, to obtain optimal values of the one or more parameters. The controller may be configured to set automatically controllable ones of the one or more parameters of the respiratory therapy system to the respective optimal values of the automatically controllable ones of the one or more parameters. The controller may be configured to generate a recommendation to a user, via an interface of the respiratory therapy system, with optimal values of manually controllable ones of the one or more parameters of the respiratory therapy system.

Some forms of the present technology may include apparatus. The apparatus may include means for generating a flow of air. The apparatus may include means for delivering the flow of air to an entrance to an airway of a patient. The apparatus may include means for conducting the flow of air between the means for generating and the means for delivering. The apparatus may include means for inserting supplementary oxygen into the flow of air. The apparatus may include means for optimising one or more parameters of the apparatus with respect to an oxygen performance metric of the apparatus, to obtain optimal values of the one or more parameters. The apparatus may include means for setting automatically controllable ones of the one or more parameters of the apparatus to the respective optimal values of the automatically controllable ones of the one or more parameters. The apparatus may include means for generating a recommendation to a user with the optimal values of manually controllable ones of the one or more parameters of the apparatus.

Some forms of the present technology may include a trigger module for a portable oxygen concentrator. The trigger module may include a housing. The housing may include an interior configured to be pneumatically connected to an outlet of the portable oxygen concentrator. The trigger module may include a piston within the housing configured to produce, when actuated, a drop in pressure within the interior. The trigger module may include a solenoid configured, when energised, to actuate the piston.

In some versions, the drop in pressure may include a pneumatic pseudo-trigger capable of triggering the release of a bolus of oxygen from the portable oxygen concentrator when detected by a pneumatic sensor of the oxygen concentrator. The trigger module may further include a spring mechanism configured to urge the piston toward its un-actuated position. The solenoid may be configured to energise in response to a pseudo-trigger command generated by an external respiratory therapy device.

Some forms of the present technology may include a method of one or more processors for computing an oxygen performance metric of a respiratory therapy system with supplementary oxygen. The method of the one or more processors may include deriving an estimate of a respiratory flow rate of a patient from a height of the patient. The method of the one or more processors may include deriving an estimate of a vent flow rate of the respiratory therapy system with supplementary oxygen using one or more parameters of the respiratory therapy and the estimated respiratory flow rate. The method of the one or more processors may include computing the oxygen performance metric using one or more parameters of an air circuit of the respiratory therapy system with supplementary oxygen, the estimated vent flow rate, a respiratory therapy device flow rate, a flow rate of the supplementary oxygen, and the estimated respiratory flow rate.

In some versions, the method of the one or more processors may further include determining the supplementary oxygen flow rate using parameters of the supplementary oxygen. The respiratory therapy may be a respiratory pressure therapy. The method may further include estimating the respiratory therapy device flow rate using the estimated respiratory flow rate and the one or more respiratory therapy parameters. The respiratory therapy may be a flow therapy. The method may include determining the respiratory device flow rate using the one or more respiratory therapy parameters.

Some forms of the present technology may include a respiratory therapy system with supplementary oxygen. The system may include a respiratory therapy device configured to generate a flow of air according to one or more therapy parameters and adapted to pneumatically couple with (a) a patient interface configured to deliver the flow of air to an entrance to an airway of a patient, (b) an air circuit configured to conduct the flow of air between the respiratory therapy device and the patient interface, and (c) an oxygen source configured to insert supplementary oxygen into the flow of air. The system may include a controller. The controller may be configured to derive an estimate of a respiratory flow rate of the patient from a height of the patient. The controller may be configured to derive an estimate a vent flow rate of the respiratory therapy system with supplementary oxygen using the one or more therapy parameters and the estimated respiratory flow rate. The controller may be configured to compute an oxygen performance metric of the respiratory therapy system with supplementary oxygen using one or more parameters of the air circuit, the estimated vent flow rate, a flow rate of the generated flow of air, a flow rate of the inserted supplementary oxygen, and the estimated respiratory flow rate.

Some forms of the present technology may include apparatus. The apparatus may include means for generating a flow of air according to one or more therapy parameters. The apparatus may include means for generating a flow of air according to one or more therapy parameters. The apparatus may include means for delivering the flow of air to an entrance to an airway of a patient. The apparatus may include means for conducting the flow of air between the means for generating and the means for delivering. The apparatus may include means for inserting supplementary oxygen into the flow of air. The apparatus may include means for deriving an estimate of a respiratory flow rate of the patient from a height of the patient. The apparatus may include means for deriving an estimate of a vent flow rate of the apparatus using the one or more therapy parameters and the estimated respiratory flow rate. The apparatus may include means for computing an oxygen performance metric of the apparatus using one or more parameters of the means for conducting, the estimated vent flow rate, a flow rate of the generated flow of air, a flow rate of the inserted supplementary oxygen, and the estimated respiratory flow rate.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, respiratory failure.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

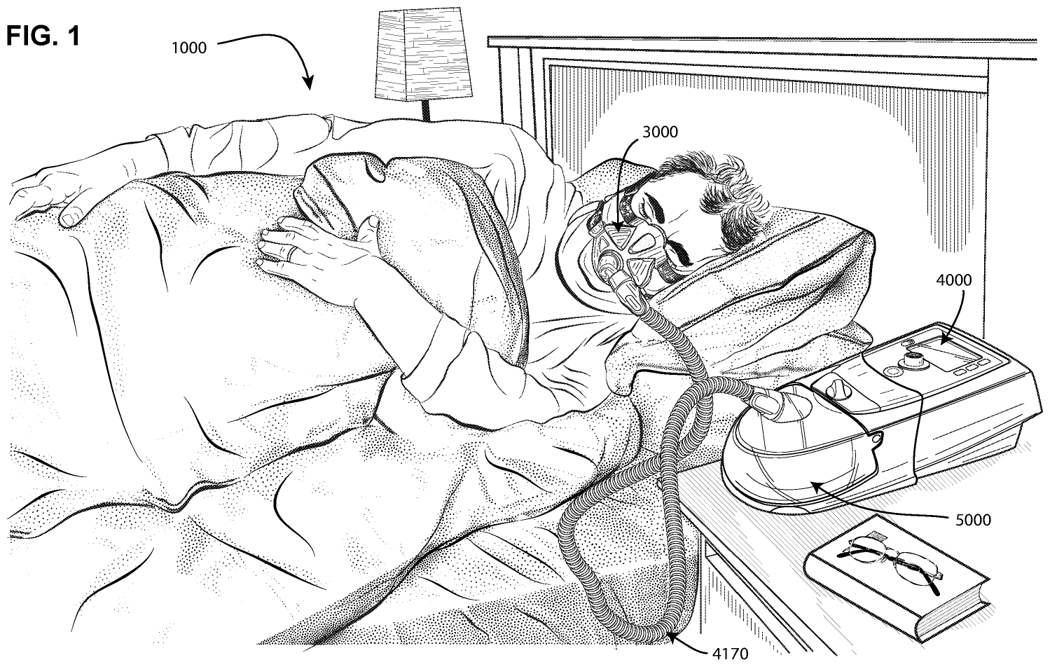

FIG. 1 shows a system including a patient 1000 wearing a patient interface 3000, in the form of a full-face mask, receiving a supply of air at positive pressure from an RPT device 4000. Air from the RPT device is conditioned in a humidifier 5000, and passes along an air circuit 4170 to the patient 1000. The patient is sleeping in a side sleeping position.

### 4.2 Respiratory System and Facial Anatomy

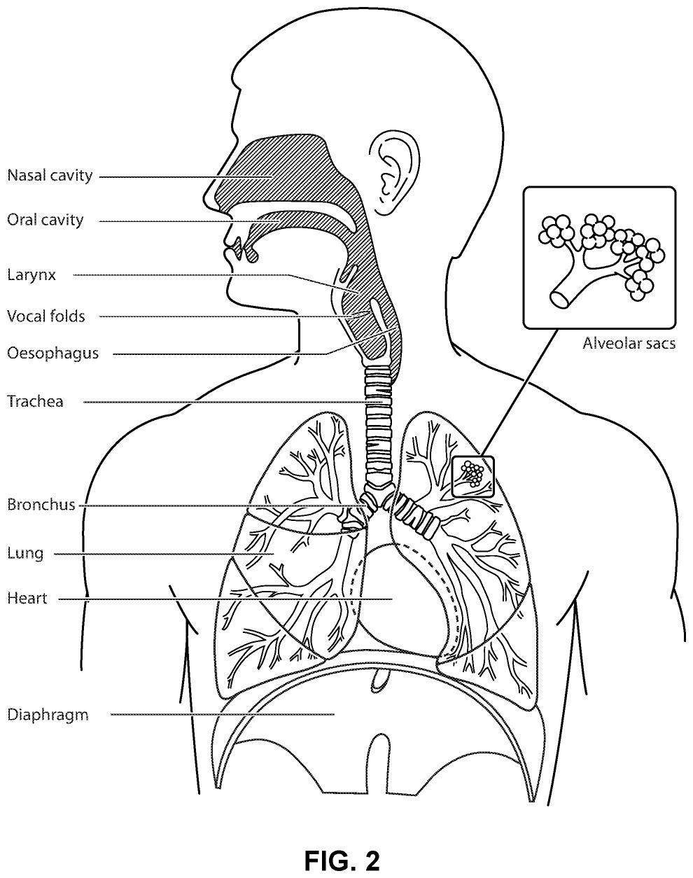

FIG. 2 shows an overview of a human respiratory system including the nasal and oral cavities, the larynx, vocal folds, oesophagus, trachea, bronchus, lung, alveoli, heart and diaphragm.

### 4.3 Patient Interface

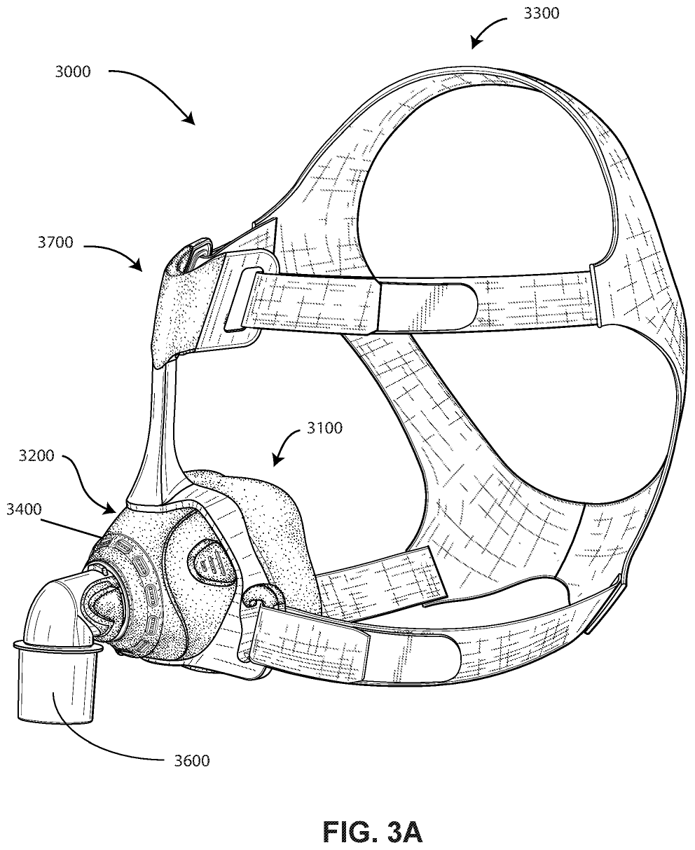

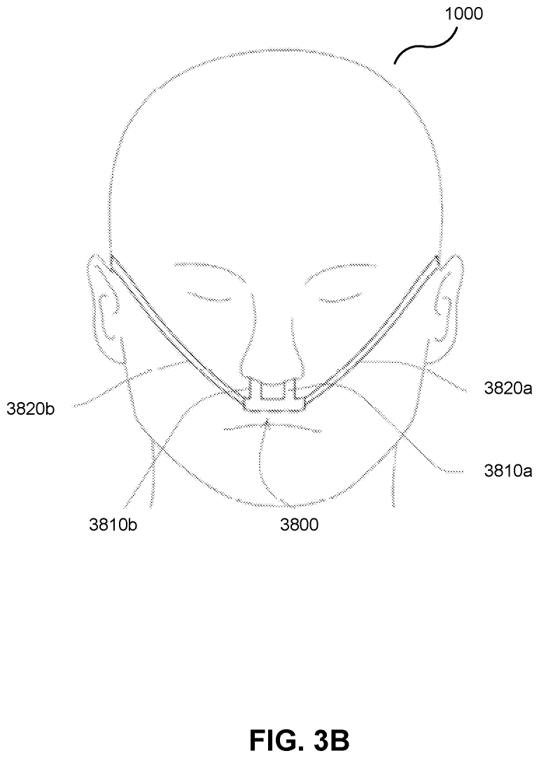

FIG. 3A shows a patient interface in the form of a nasal mask in accordance with one form of the present technology.

FIG. 3B shows a patient 1000 wearing an unsealed patient interface in the form of a nasal cannula in accordance with one form of the present technology.

### 4.4 RPT Device

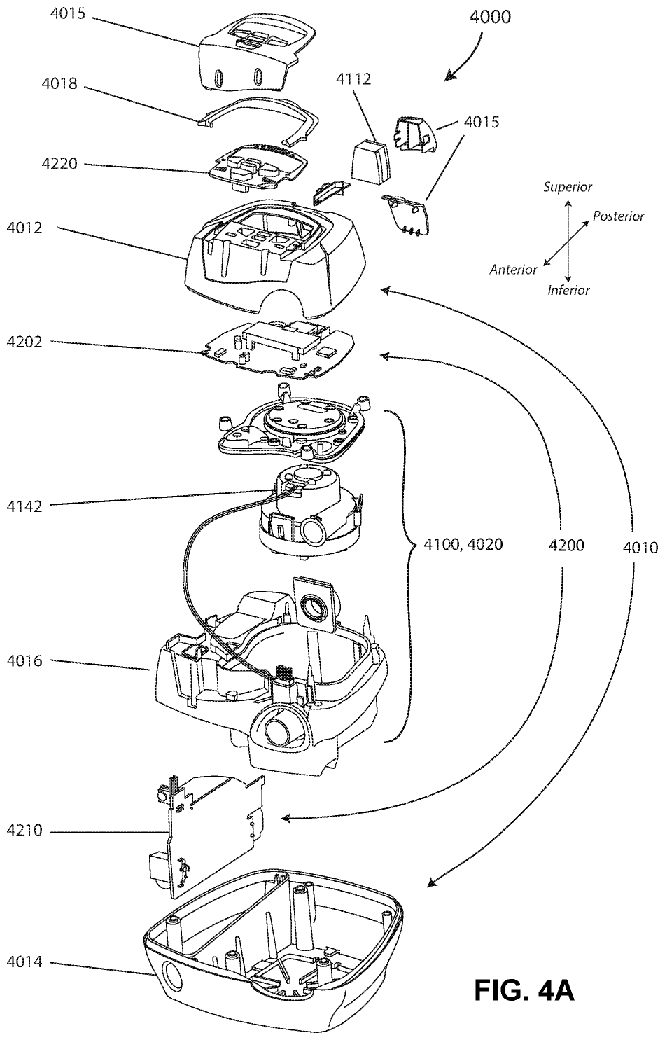

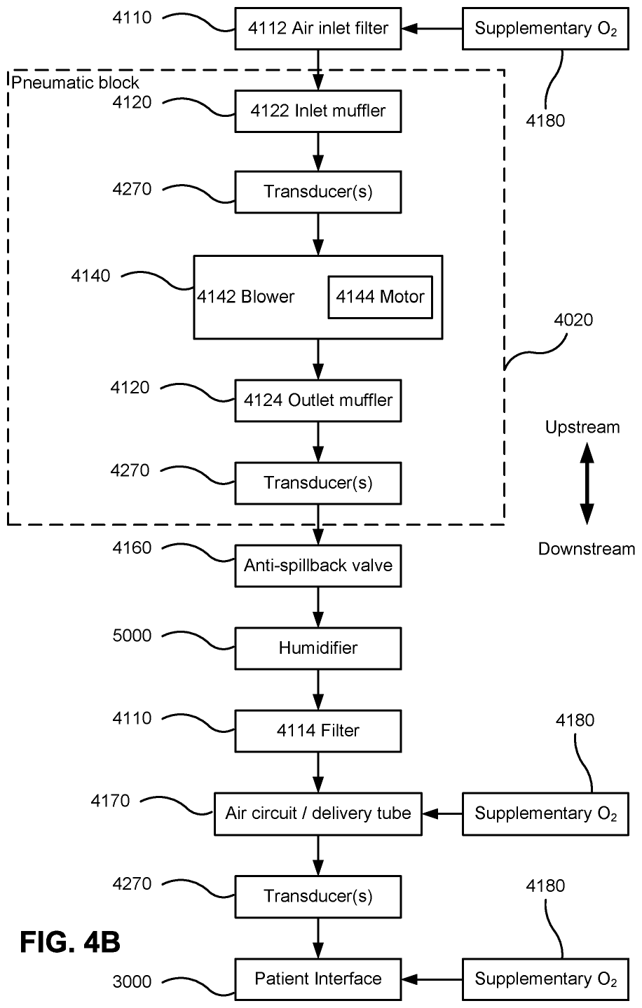

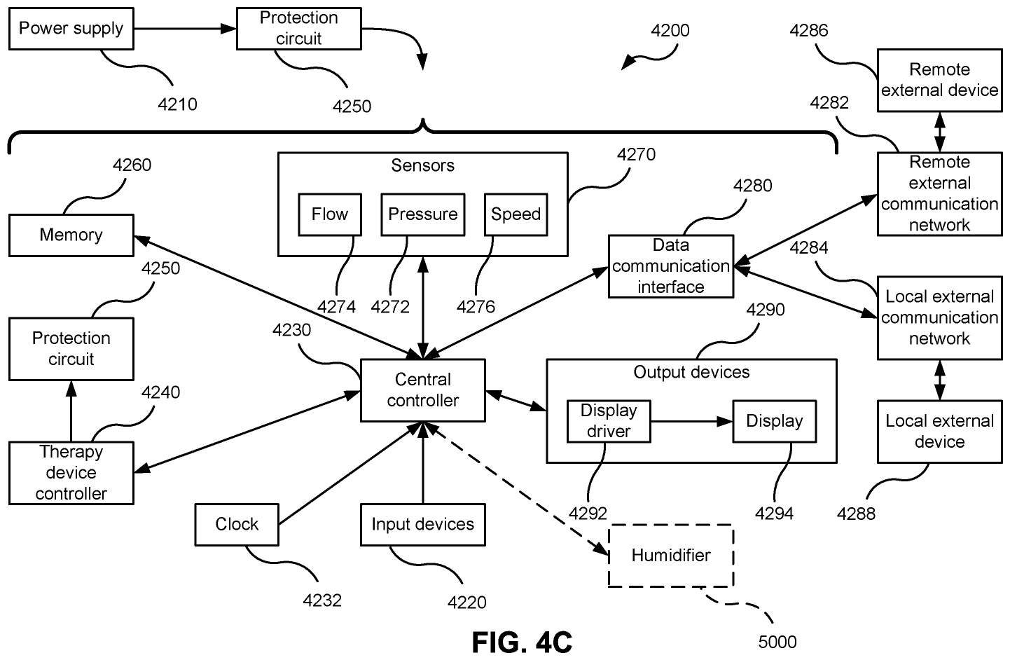

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4D is a schematic diagram of the algorithms implemented in an RPT device in accordance with one form of the present technology.

### 4.5 Humidifier

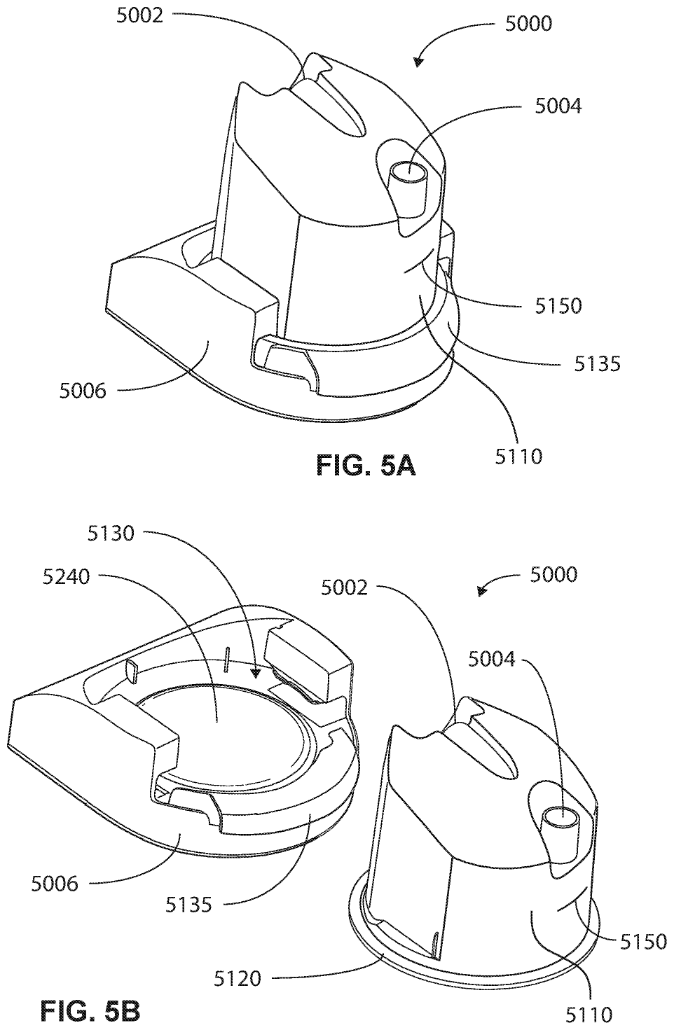

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Breathing Waveforms

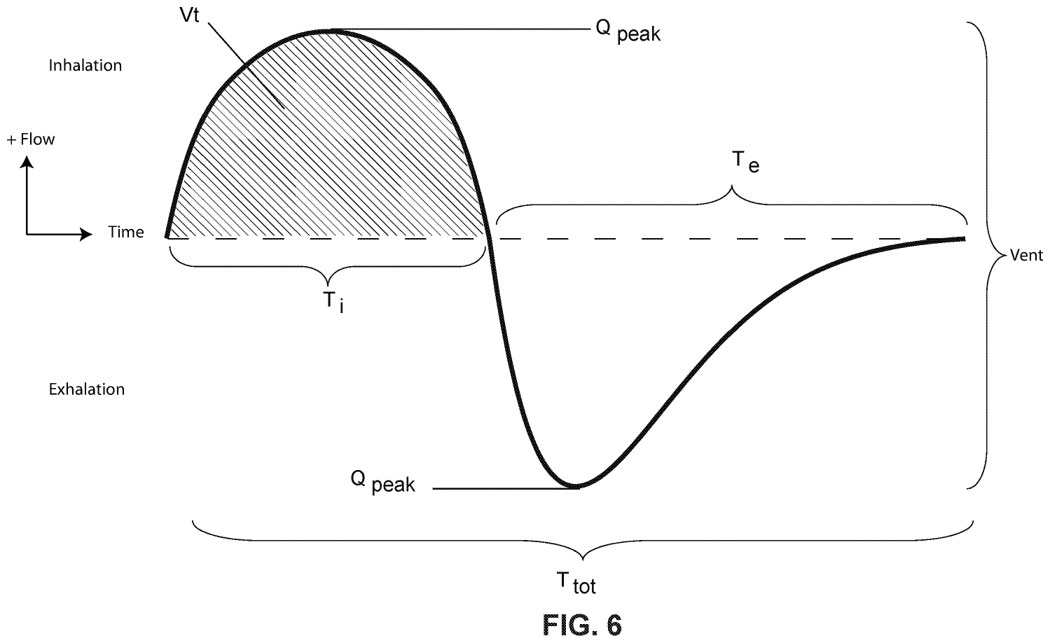

FIG. 6 shows a model typical respiratory flow rate profile of a person while sleeping.

### 4.7 Oxygen Concentrator

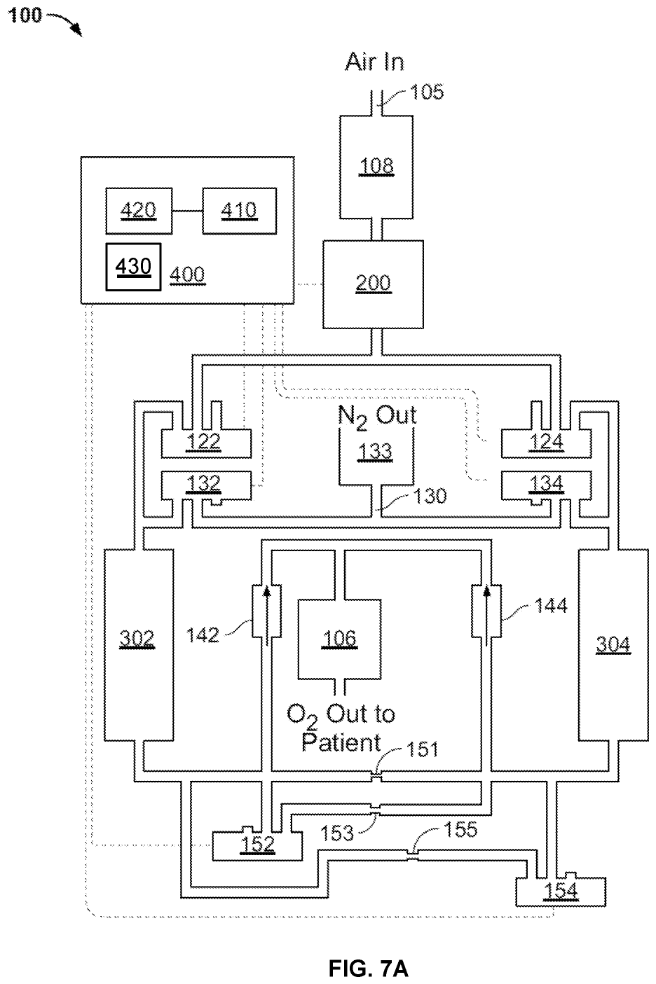

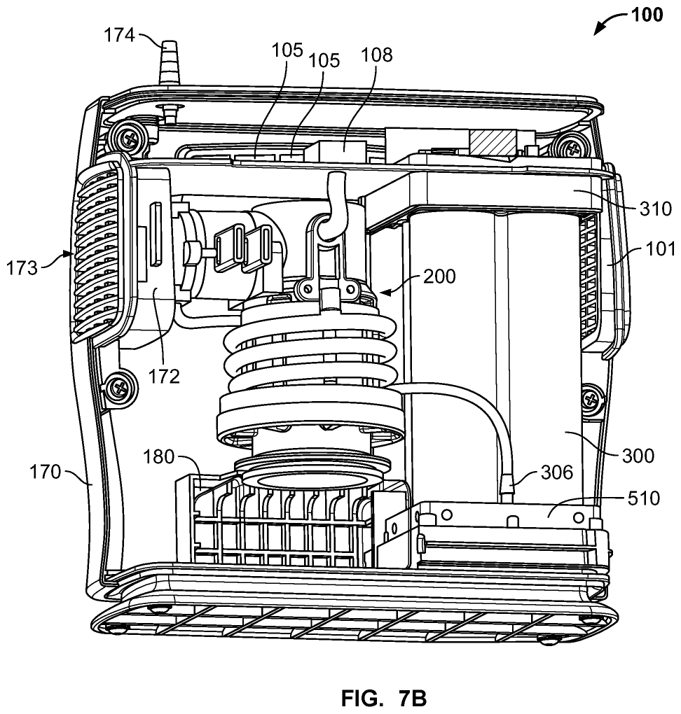

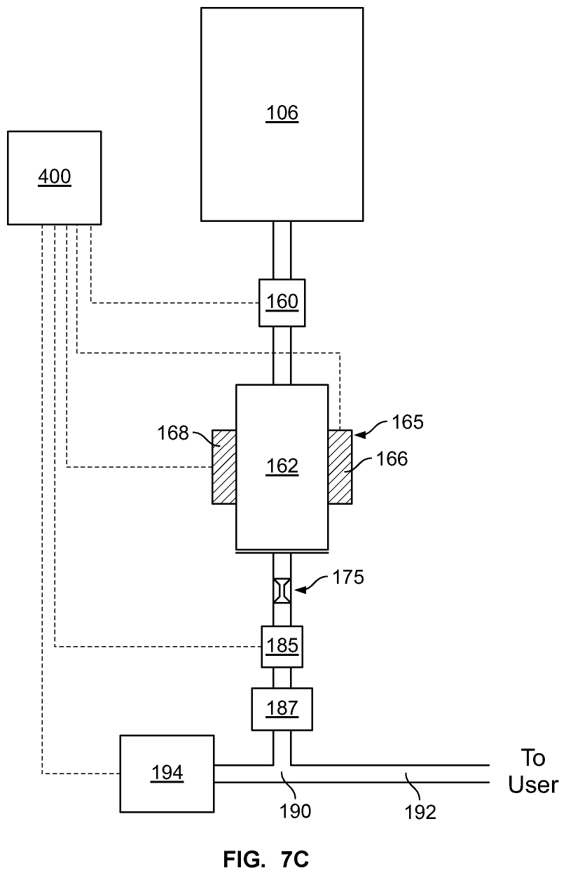

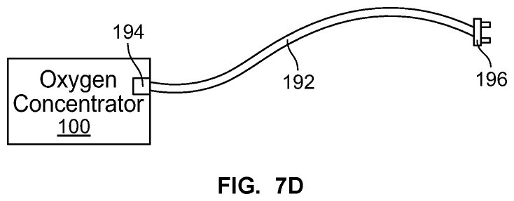

FIG. 7A is a schematic diagram of the components of an oxygen concentrator according to one form of the present technology.

FIG. 7B is a side view of the main components of the oxygen concentrator of FIG. 7A.

FIG. 7C is a schematic diagram of the outlet system of the oxygen concentrator of FIG. 7A.

FIG. 7D depicts an outlet conduit and interface for the oxygen concentrator of FIG. 7A.

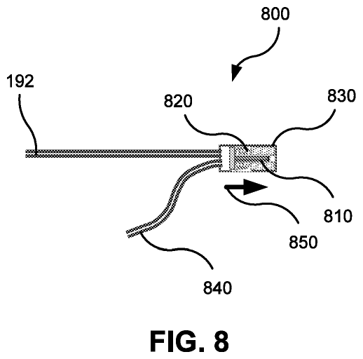

FIG. 8 is a block diagram illustrating a trigger module according to one form of the present technology.

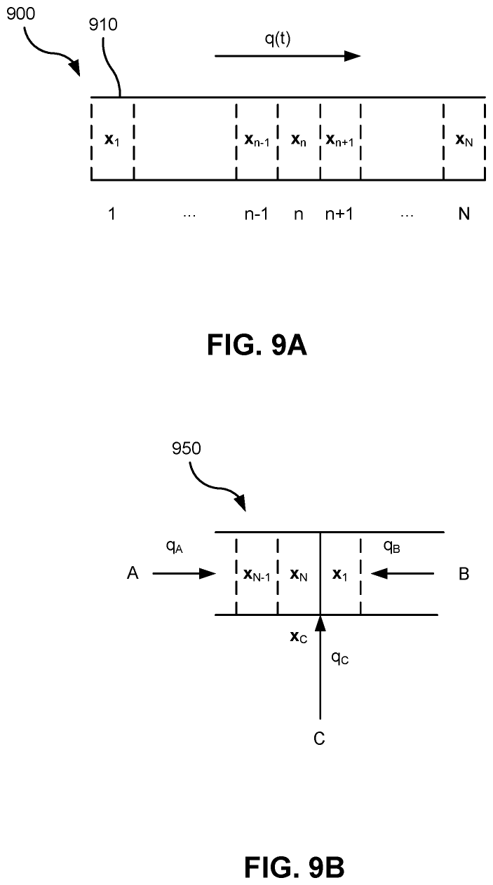

FIGS. 9A and 9B are illustrations of a model of pipe transport of gas mixtures.

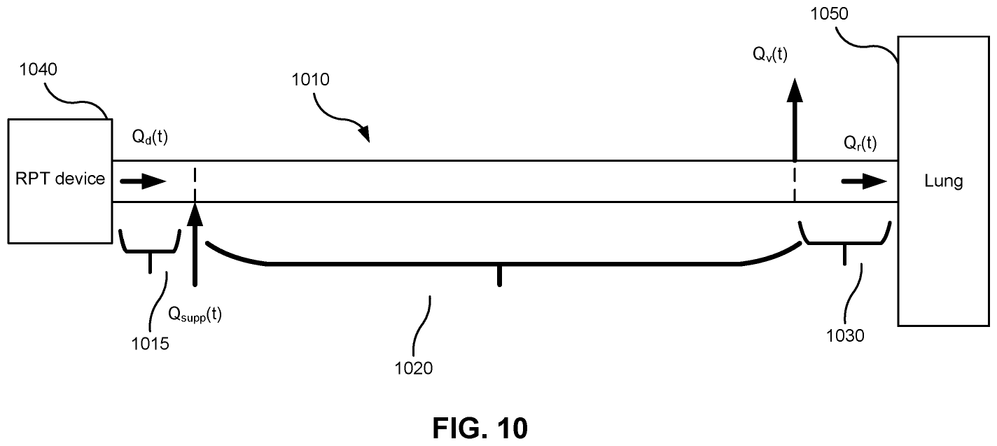

FIG. 10 is an illustration of a pipe transport model of FIGS. 9A and 9B applied to a respiratory therapy system with supplementary oxygen.

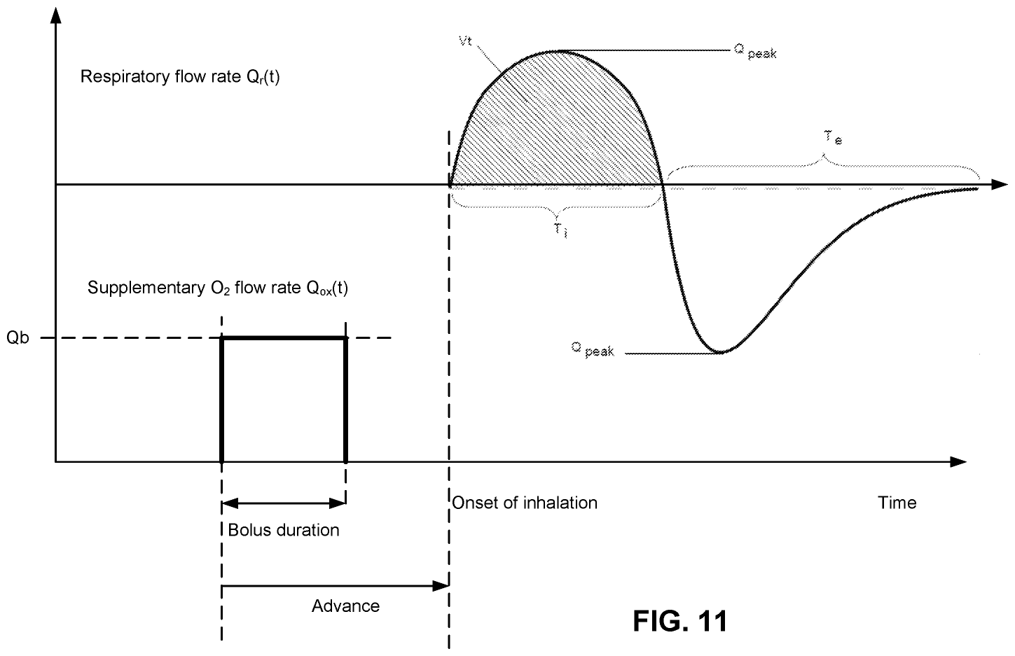

FIG. 11 is a graph illustrating respiratory flow rate and supplementary oxygen flow rate.

### 4.8 Respiratory Therapy with Supplementary Oxygen

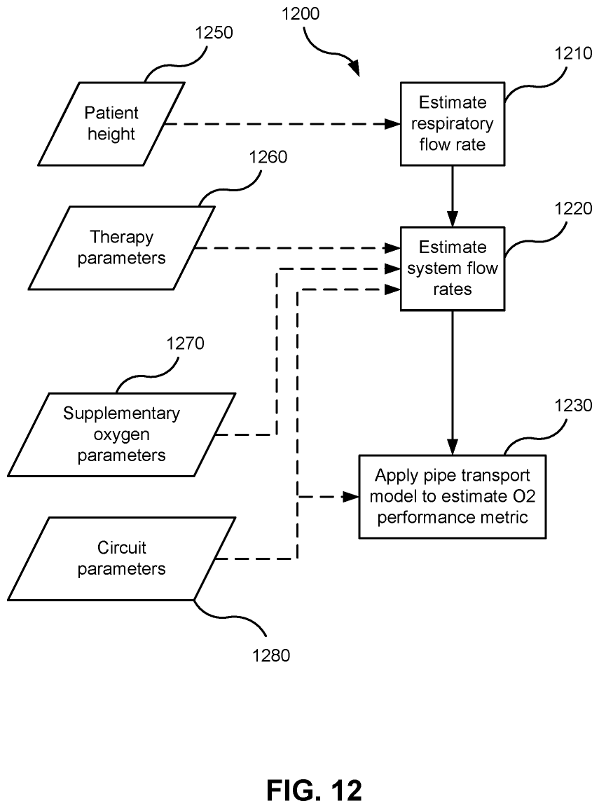

FIG. 12 is a flow chart illustrating a “theoretical” method of estimating an oxygen performance metric of a respiratory therapy system with supplementary oxygen according to one aspect of the present technology.

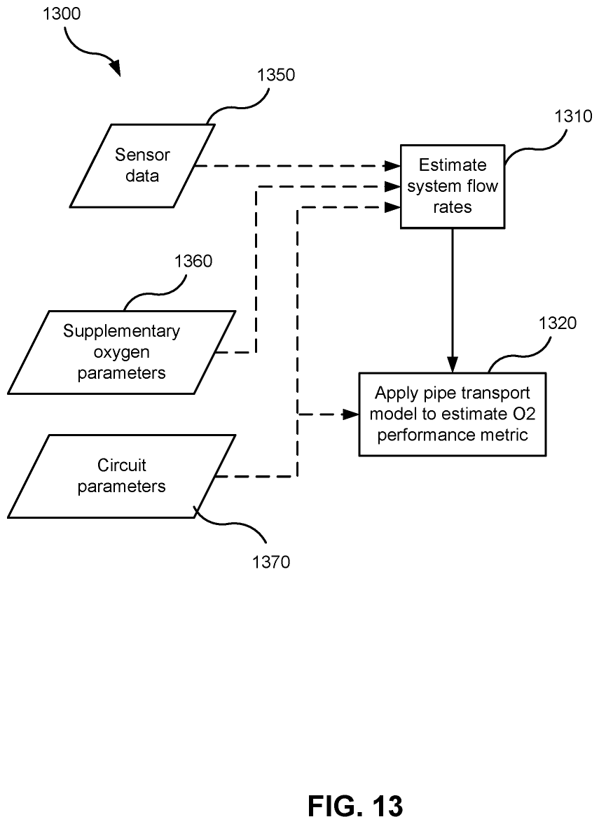

FIG. 13 is a flow chart illustrating an “empirical” method of estimating an oxygen performance metric of a respiratory therapy system with supplementary oxygen according to another aspect of the present technology.

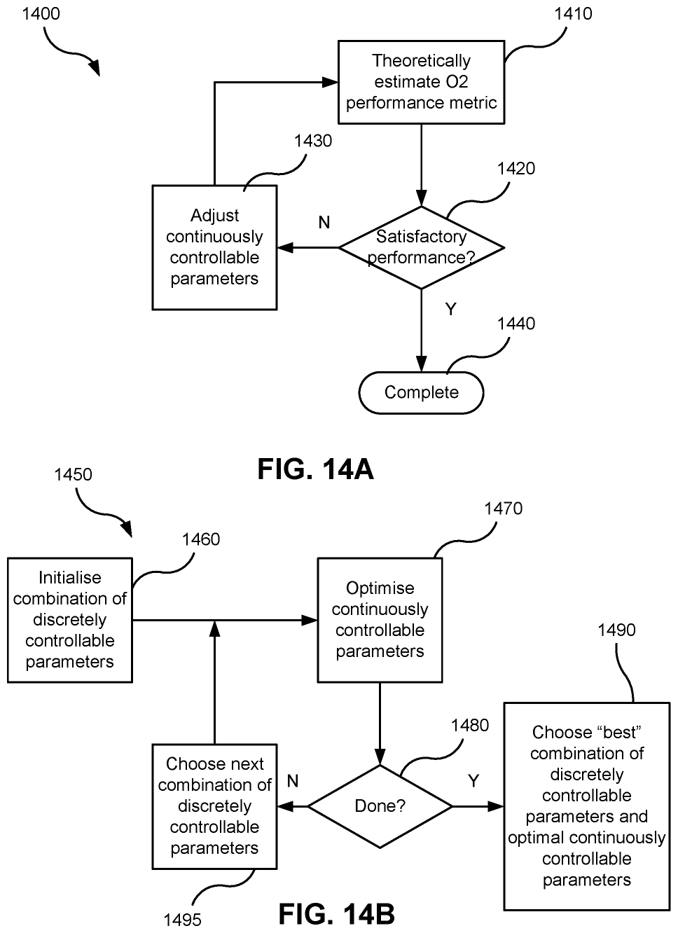

FIG. 14A is a flow chart illustrating a method of optimising continuously controllable parameters of a therapy system/patient combination according to another aspect of the present technology.

FIG. 14B is a flow chart illustrating a method of optimising controllable parameters of a therapy system/patient combination according to another aspect of the present technology.

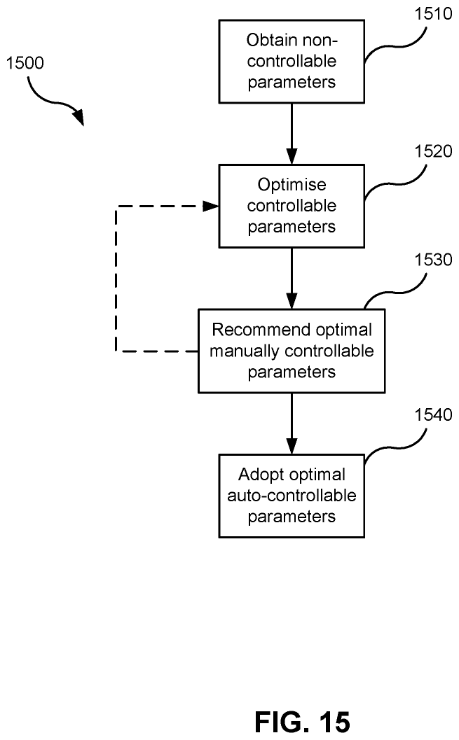

FIG. 15 is a flow chart illustrating a method of “pre-optimising” the controllable parameters of a therapy system/patient combination before the start of therapy according to another aspect of the present technology.

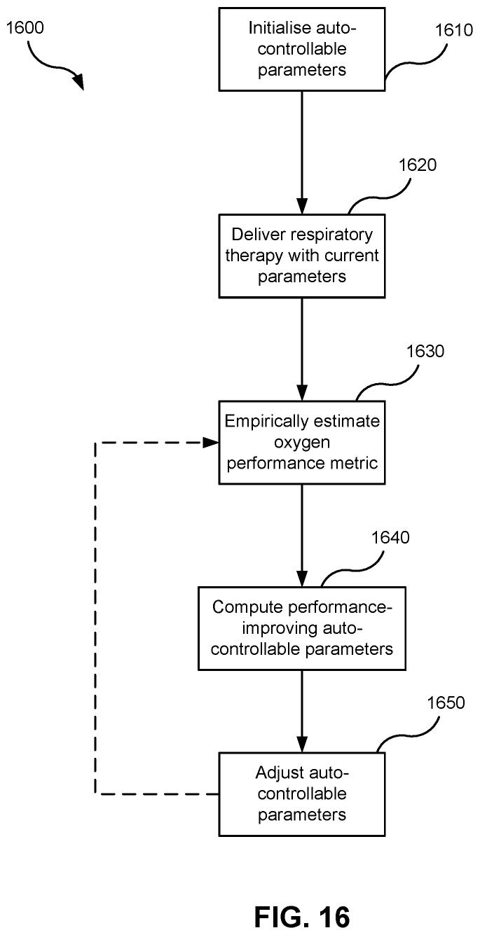

FIG. 16 is a flow chart illustrating a method of improving an oxygen performance metric of a therapy system/patient combination during respiratory therapy with supplementary oxygen according to another aspect of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

In one form, the present technology comprises a respiratory therapy system with supplementary oxygen for treating a respiratory disorder. The respiratory therapy system with supplementary oxygen may comprise an RPT device 4000 for supplying a flow of air to the patient 1000 via an air circuit 4170 to a patient interface 3000 or 3800, and an oxygen concentrator 100 to supply the supplementary oxygen into the flow of air.

### 5.1 Patient Interface

A non-invasive sealed patient interface 3000 comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use, the seal-forming structure 3100 is arranged to surround an entrance to the airways of the patient so as to facilitate the supply of air at positive pressure to the airways.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. The air to the nasal prongs may be delivered by one or more air supply lumens 3820a, 3820b that are coupled with the nasal cannula 3800. The lumens 3820a, 3820b lead from the nasal cannula 3800 to a respiratory therapy device via an air circuit. The unsealed patient interface 3800 is particularly suitable for delivery of flow therapies, in which the RPT device generates the flow of air at controlled flow rates rather than controlled pressures. The “vent” at the unsealed patient interface 3800, through which excess airflow escapes to ambient, is the passage between the end of the prongs 3810a and 3810b of the cannula 3800 via the patient's nares to atmosphere.

### 5.2 Air Circuit

An air circuit 4170 in accordance with one form of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface 3000 or 3800. In some forms, there may be separate limbs of the circuit for inhalation and exhalation. In other forms, a single limb circuit is used.

### 5.3 RPT Device

An RPT device 4000 comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of delivering a flow of air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.3.1 RPT Device Mechanical & Pneumatic Component

An RPT device may comprise one or more of the following components in an integral unit. In an alternative form, one or more of the following components may be located as respective separate units.

5.3.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000 or 3800.

5.3.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.3.1.3 Pressure Generator

In one form of the present technology, a pressure generator 4140 for producing a flow, or a supply, of air at positive pressure is a controllable blower 4142. For example the blower 4142 may include a brushless DC motor 4144 with one or more impellers housed in a blower housing, such as in a volute. The blower may be capable of delivering a supply of air, for example at a rate of up to about 120 litres/minute, at a positive pressure in a range from about 4 cmH₂O to about 20 cmH₂O, or in other forms up to about 30 cmH₂O. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 is under the control of the therapy device controller 4240.

In other forms, a pressure generator 4140 may be a piston-driven pump, a pressure regulator connected to a high pressure source (e.g. compressed air reservoir), or a bellows.

### 5.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form, a signal from a transducer 4270 may be filtered, such as by low-pass, high-pass or band-pass filtering.

### 5.3.1.4.1 Flow Rate Sensor

A flow rate sensor 4274 in accordance with the present technology may be based on a differential pressure transducer, for example, an SDP600 Series differential pressure transducer from SENSIRION.

In one form, a signal representing a flow rate of the flow of air at the output of the RPT device 4000 is generated by the flow rate sensor 4274.

### 5.3.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal representing a pressure of the flow of air at the output of the RPT device 4000 (the device pressure) is generated by the pressure sensor 4272.

### 5.3.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.3.1.6 Supplementary Gas Delivery

In one form of the present technology, supplementary gas, e.g. oxygen 4180 is delivered to one or more points in the pneumatic path, such as upstream of the pneumatic block 4020, to a point in the air circuit 4170, and/or at the patient interface 3000 or 3800.

### 5.3.2 RPT Device Electrical Components

### 5.3.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.3.2.3 Central Controller

In one form of the present technology, the central controller 4230 is one or a plurality of processors suitable to control an RPT device 4000.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000.

The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.3.2.4 Clock

The RPT device 4000 may include a clock 4232 that is connected to the central controller 4230.

### 5.3.2.5 Therapy Device Controller

In one form of the present technology, therapy device controller 4240 is a therapy control module 4330 that forms part of the algorithms executed by the central controller 4230.

In one form of the present technology, therapy device controller 4240 is a dedicated motor control integrated circuit. For example, in one form a MC33035 brushless DC motor controller, manufactured by ONSEMI is used.

### 5.3.2.6 Protection Circuits

The one or more protection circuits 4250 in accordance with the present technology may comprise an electrical protection circuit, a temperature and/or pressure safety circuit.

### 5.3.2.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms.

### 5.3.2.8 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the local external communication network 4284 or the remote external communication network 4282.

In one form, local external communication network 4284 utilises one or more communication standards, such as Bluetooth, or a consumer infrared protocol.

In one form, remote external device 4286 is one or more computers, for example a server or a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet, remote control, portable oxygen concentrator, or other ancillary device.

### 5.3.2.9 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.3.2.9.1 Display Driver

A display driver 4292 receives as an input the characters, symbols, or images intended for display on the display 4294, and converts them to commands that cause the display 4294 to display those characters, symbols, or images.

### 5.3.2.9.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.3.3 RPT Device Algorithms

As mentioned above, in some forms of the present technology, the central controller 4230 may be configured to implement one or more algorithms expressed as computer programs stored in a non-transitory computer readable storage medium, such as memory 4260.

In other forms of the present technology, some portion or all of the algorithms may be implemented by a controller of an external device such as the local external device 4288 or the remote external device 4286. In such forms, data representing the input signals and/or intermediate algorithm outputs necessary for the portion of the algorithms to be executed at the external device may be communicated to the external device via the local external communication network 4284 or the remote external communication network 4282. In such forms, the portion of the algorithms to be executed at the external device may be expressed as computer programs stored in a non-transitory computer readable storage medium accessible to the controller of the external device. Such programs configure the controller of the external device to execute the portion of the algorithms to be executed at the external device.

In general, each algorithm receives as an input a signal from a transducer 4270, for example a flow rate sensor 4274 or a pressure sensor 4272, and performs one or more process steps to calculate one or more output values that may be used as an input to another algorithm.

### 5.3.3.1 Pressure Therapy Algorithms

### 5.3.3.1.1 Pressure Drop Estimation

In one form of the present technology, an pressure drop estimation algorithm receives as an input a signal from the flow rate sensor 4274 representative of the flow rate of the airflow leaving the RPT device 4000 (the device flow rate Qd) and estimates the pressure drop ΔP through the air circuit 4170. The dependence of the pressure drop ΔP on the flow rate Q may be modelled for the particular air circuit 4170 by a pressure drop characteristic ΔP(Q)

### 5.3.3.1.2 Vent Flow Rate Estimation

In one form of the present technology, a vent flow rate estimation algorithm receives as inputs a signal from the pressure sensor 4272 representative of the pressure of the airflow leaving the RPT device 4000 (the device pressure Pd) and the pressure drop ΔP through the air circuit 4170, and estimates a vent flow rate of air, Qv, from a vent 3400 in a patient interface 3000 or 3800. The pressure, Pm, in the patient interface 3000 or 3800 may be estimated as the device pressure Pd minus the air circuit pressure drop ΔP. The dependence of the vent flow rate Qv on the interface pressure Pm for the particular vent 3400 in use may be modelled by a vent characteristic Qv(Pm).

### 5.3.3.1.3 Respiratory Flow Rate Estimation

In one form of the present technology, a respiratory flow rate estimation algorithm receives as inputs a signal from the flow rate sensor 4274 representative of the device flow rate Qd, and a vent flow rate Qv, and estimates a respiratory flow rate of air Qr inspired by the patient by subtracting the vent flow rate Qv from the device flow rate Qd (represented by a signal from the flow rate sensor 4274).

### 5.3.3.1.4 Respiratory Phase Determination

In one form of the present technology, a phase determination algorithm receives as an input a signal indicative of respiratory flow rate, Qr, and provides as an output a phase of a current breathing cycle of the patient 1000.

One implementation of phase determination provides a bi-valued phase output with values of either inhalation or exhalation, for example represented as values of 0 and 0.5 revolutions respectively, upon detecting the onset of inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold.

By measuring the intervals between adjacent onsets of inhalation, the patient's total breath time Ttot may be estimated. By measuring the intervals between onsets of inhalation and the following onsets of exhalation, the patient's inspiratory time Ti may be estimated. By measuring the intervals between onsets of exhalation and the following onsets of inhalation, the patient's expiratory time Te may be estimated. Having an estimate of expiratory time Te allows the onset of inhalation to be predicted to occur one expiratory time Te after the onset of exhalation.

### 5.3.3.1.5 Device Flow Rate Estimation Algorithm

In one form of the present technology, a device flow rate estimation algorithm receives as inputs the respiratory flow rate Qr and the treatment pressure profile Pt(t), and estimates the device flow rate Qd of the RPT device 4000. The device flow rate estimation algorithm first estimates the vent flow rate Qv from the interface pressure Pm, which is (as described below) approximately equal to the treatment pressure Pt, using the vent characteristic Qv(Pm) of the vent 3400. The device flow rate estimation algorithm then estimates the device flow rate Qd as the sum of the respiratory flow rate Qr and the vent flow rate Qv.

### 5.3.3.2 Flow Therapy Algorithms

### 5.3.3.2.1 Respiratory Flow Rate Estimation

In one form of the present technology, a respiratory flow rate estimation algorithm receives as inputs a signal from the flow rate sensor 4274 representative of the device flow rate Qd, a signal from the pressure sensor 4272 representative of the device pressure Pd, and estimates the respiratory flow rate of air Qr inspired by the patient using the pressure drop characteristic ΔP(Q) of the air circuit 4170. One such algorithm is disclosed in the co-pending U.S. provisional application No. 62/832,091, filed 10 Apr. 2019.

### 5.4 Humidifier

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.5 Breathing Waveforms

FIG. 6 shows a model typical respiratory flow rate profile of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate −0.5 L/s. The total duration of the breath, Ttot, is about 4 s. The person typically breathes at a rate of about 15 breaths per minute (BPM), with ventilation Vent about 7.5 L/min. A typical duty cycle, the ratio of Ti to Ttot, is about 40%.

### 5.6 Respiratory Therapy Modes

Various respiratory therapy modes may be implemented by the disclosed respiratory therapy system.

### 5.6.1 CPAP Therapy

In some forms of respiratory pressure therapy, the central controller 4230 holds the treatment pressure Pt (which represents a target value to be achieved by the interface pressure Pm at the current instant of time) constant throughout the respiratory cycle. Such forms are generally grouped under the heading of CPAP therapy. In CPAP therapy, the treatment pressure may be a constant value that is hard-coded or manually entered to the RPT device 4000. Alternatively, the central controller 4230 may repeatedly compute the treatment pressure as a function of indices or measures of sleep disordered breathing.

### 5.6.2 Bi-Level Therapy

In other forms of respiratory pressure therapy, the central controller 4230 oscillates the treatment pressure Pt between two values or levels in synchrony with the spontaneous respiratory effort of the patient 1000. That is, the central controller 4230 increases, or starts increasing, the treatment pressure to or toward a maximum value known as the IPAP at the onset of inspiration, and decreases, or starts decreasing, the treatment pressure Pt to or toward a minimum pressure known as the EPAP at the start of expiration. The difference between the IPAP and the EPAP is the amplitude A of the oscillation.

In some forms of bi-level therapy, the IPAP is a treatment pressure that has the same purpose as the treatment pressure in CPAP therapy modes, and the EPAP is the IPAP minus a “small” value (a few cmH₂O) sometimes referred to as the Expiratory Pressure Relief (EPR). Such forms are sometimes referred to as CPAP therapy with EPR, which is generally thought to be more comfortable than straight CPAP therapy. In CPAP therapy with EPR, either or both of the IPAP and the EPAP may be constant values that are hard-coded or manually entered to the RPT device 4000. Alternatively, a therapy parameter determination algorithm may repeatedly compute the IPAP and/or the EPAP during CPAP with EPR. In this alternative, the therapy parameter determination algorithm repeatedly computes the EPAP and/or the IPAP as a function of indices or measures of sleep disordered breathing.

In other forms of bi-level therapy, the amplitude A is large enough that the RPT device 4000 does some or all of the work of breathing of the patient 1000. In such forms, known as pressure support ventilation therapy, the amplitude A is referred to as the pressure support, or swing.

In some forms of pressure support ventilation therapy, known as fixed pressure support ventilation therapy, the pressure support A is fixed at a predetermined value, e.g. 10 cmH₂O. The predetermined pressure support value is a setting of the RPT device 4000, and may be set for example by hard-coding during configuration of the RPT device 4000 or by manual entry through the input device 4220. In other forms, the pressure support A may be variable by the central controller 4230 during therapy to achieve some therapeutic goal such as stability of breathing or delivery of a predetermined tidal volume. Likewise, the IPAP and/or the EPAP may be fixed or variable during therapy.

### 5.6.3 High Flow Therapy

In other forms of respiratory therapy, the pressure of the flow of air is not controlled as it is for respiratory pressure therapy. Rather, the central controller 4230 controls the pressure generator 4140 to deliver a flow of air whose flow rate Qd is controlled to a treatment or target flow rate Qt that is typically positive throughout the patient's breathing cycle. Such forms are generally grouped under the heading of flow therapy. In flow therapy, the treatment flow rate Qt may be a constant value that is hard-coded or manually entered to the RPT device 4000. If the treatment flow rate Qt is sufficient to exceed the patient's peak inspiratory flow rate, the therapy is generally referred to as high flow therapy (HFT). Alternatively, the treatment flow rate may be a profile Qt(t) that varies in synchrony with the respiratory cycle.

### 5.7 Portable Oxygen Concentrator

Oxygen concentrators typically take advantage of pressure swing adsorption (PSA). Pressure swing adsorption may involve using a compressor to increase gas pressure inside a canister that contains particles of a gas separation adsorbent. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The gas separation adsorbent may be regenerated by reducing the pressure, which reverses the adsorption of molecules from the adsorbent. Further details regarding oxygen concentrators may be found, for example, in U.S. patent application Ser. No. 12/163,549, published Mar. 12, 2009 as U.S. Publication No. 2009-0065007, entitled “Oxygen Concentrator Apparatus and Method”, and incorporated herein by reference.

Ambient air usually includes approximately 78% nitrogen and 21% oxygen with the balance comprised of argon, carbon dioxide, water vapour, and other trace gases. If a gas mixture such as air, for example, is passed under pressure through a vessel containing a gas separation adsorbent bed that attracts nitrogen more strongly than it does oxygen, part or all of the nitrogen will stay in the bed, and the gas coming out of the vessel will be enriched in oxygen. When the bed reaches the end of its capacity to adsorb nitrogen, it can be regenerated by reducing the pressure, thereby releasing the adsorbed nitrogen. It is then ready for another cycle of producing oxygen enriched gas. By alternating canisters in a two-canister system, one canister can be collecting oxygen while the other canister is being purged (resulting in a continuous separation of the oxygen from the nitrogen). In this manner, oxygen can be accumulated out of the air for a variety of uses include providing supplementary oxygen to patients.

FIG. 7A contains a schematic diagram of components of a portable oxygen concentrator 100, according to one form of the present technology. Oxygen concentrator 100 may concentrate oxygen out of an air stream to provide oxygen enriched gas to a patient. As used herein, “oxygen enriched gas” is composed of at least about 50% oxygen, at least about 60% oxygen, at least about 70% oxygen, at least about 80% oxygen, at least about 90% oxygen, at least about 95% oxygen, at least about 98% oxygen, or at least about 99% oxygen.

Portable oxygen concentrator 100 may have a weight and size that allows the oxygen concentrator to be carried by hand and/or in a carrying case. As examples, oxygen concentrator 100 has a weight of less than about 20 lbs, less than about 15 lbs, less than about 10 lbs, or less than about 5 lbs. As examples, oxygen concentrator 100 has a volume of less than about 1000 cubic inches, less than about 750 cubic inches, less than about 500 cubic inches, less than about 250 cubic inches, or less than about 200 cubic inches.

Oxygen may be collected from ambient air by pressurising ambient air in canisters 302 and 304, which include a gas separation adsorbent. Gas separation adsorbents useful in an oxygen concentrator are capable of separating at least nitrogen from an air stream to produce oxygen enriched gas. Examples of gas separation adsorbents include molecular sieves that are capable of separation of nitrogen from an air stream. Examples of adsorbents that may be used in an oxygen concentrator include, but are not limited to, zeolites (natural) or synthetic crystalline aluminosilicates that separate nitrogen from oxygen in an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 7A, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In one form, an inlet muffler 108 may be coupled to air inlet 105 to reduce sound produced by air being pulled into the oxygen concentrator by compression system 200. In one form, inlet muffler 108 may be a moisture and sound absorbing muffler. For example, a water absorbent material (such as a polymer water absorbent material or a zeolite material) may be used to both absorb water from the incoming air and to reduce the sound of the air passing into the air inlet 105.

Compression system 200 may include one or more compressors capable of compressing air. Pressurized air, produced by compression system 200, may be forced into one or both of the canisters 302 and 304. In some forms, the ambient air may be pressurized in the canisters to a pressure approximately in a range of 13-20 pounds per square inch gauge pressure (psig). Other pressures may also be used, depending on the type of gas separation adsorbent disposed in the canisters.

Coupled to each canister 302/304 are inlet valves 122/124 and outlet valves 132/134. As shown in FIG. 7A, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some forms, inlet valves 122/124 and outlet valves 132/134 may be silicon plunger solenoid valves. Other types of valves, however, may be used. Plunger valves offer advantages over other kinds of valves by being quiet and having low slippage.

In one form, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. A controller 400 is electrically coupled to valves 122, 124, 132, and 134. Controller 400 includes one or more processors 410 operable to execute program instructions stored in memory 420. The program instructions are adapted to configure the controller 400 to perform various predefined methods that are used to operate the oxygen concentrator 100. Memory 420 may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some forms, the voltages and the duration of the voltages used to open the input and output valves may be controlled by controller 400.

The controller 400 may include a transceiver 430 that may communicate with external devices to transmit data collected by the processor 410 or receive instructions from an external computing device for the processor 410.

Check valves 142 and 144 are coupled to canisters 302 and 304, respectively. Check valves 142 and 144 may be one way valves that are passively operated by the pressure differentials that occur as the canisters are pressurized and vented, or may be active valves. Check valves 142 and 144 are coupled to canisters to allow oxygen produced during pressurization of the canisters to flow out of the canister, and to inhibit back flow of oxygen or any other gases into the canisters. In this manner, check valves 142 and 144 act as one way valves allowing oxygen enriched gas to exit the respective canisters during pressurization.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In one form, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched gas production. If the break pressure for reverse flow is reduced or set too low, there is, generally, a reduction in oxygen enriched gas pressure.

In one form, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 while canister 302 is pressurized. Canister 302 is pressurized until the pressure in canister is sufficient to open check valve 142. Oxygen enriched gas produced in canister 302 exits through check valve and, in one form, is collected in accumulator 106.

After some time, the gas separation adsorbent will become saturated with nitrogen and will be unable to separate significant amounts of nitrogen from incoming air. This point is usually reached after a predetermined time of oxygen enriched gas production. In the form of the present technology described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched gas in the same manner described above. Pressurization of canister 304 is achieved by closing outlet valve 134 and opening inlet valve 124. The oxygen enriched gas exits canister 304 through check valve 144.

During venting of canister 302, outlet valve 132 is opened allowing pressurized gas (mainly nitrogen) to exit the canister through concentrator outlet 130. In one form, the vented gases may be directed through muffler 133 to reduce the noise produced by releasing the pressurized gas from the canister. As gas is released from canister 302, the pressure in the canister drops, allowing the nitrogen to become desorbed from the gas separation adsorbent. The released nitrogen exits the canister through outlet 130, resetting the canister to a state that allows renewed separation of oxygen from an air stream. Muffler 133 may include open cell foam (or another material) to muffle the sound of the gas leaving the oxygen concentrator. In some forms, the combined muffling components/techniques for the input of air and the output of gas, may provide for oxygen concentrator operation at a sound level below 50 decibels.

During venting of the canisters, it is advantageous that at least a majority of the nitrogen is removed. In one form, at least about 50%, at least about 60%, at least about 70%, at least about 80%, at least about 90%, at least about 95%, at least about 98%, or substantially all of the nitrogen in a canister is removed before the canister is re-used to separate oxygen from air. In some forms, a canister may be further purged of nitrogen using an oxygen enriched stream that is introduced into the canister from the other canister.

In one form, a portion of the oxygen enriched gas may be transferred from canister 302 to canister 304 when canister 304 is being vented of nitrogen. Transfer of oxygen enriched gas from canister 302 to 304, during venting of canister 304, helps to further purge nitrogen (and other gases) from the canister. In one form, oxygen enriched gas may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some forms, the flow restrictors may be press fit flow restrictors that restrict air flow by introducing a narrower diameter in their respective tubes. In some forms, the press fit flow restrictors may be made of sapphire, metal or plastic (other materials are also contemplated).

Flow of oxygen enriched gas is also controlled by use of valve 152 and valve 154. Valves 152 and 154 may be opened for a short duration during the venting process (and may be closed otherwise) to prevent excessive oxygen loss out of the purging canister. Other durations are also contemplated. In one form, canister 302 is being vented and it is desirable to purge canister 302 by passing a portion of the oxygen enriched gas being produced in canister 304 into canister 302. A portion of oxygen enriched gas, upon pressurization of canister 304, will pass through flow restrictor 151 into canister 302 during venting of canister 302. Additional oxygen enriched gas is passed into canister 302, from canister 304, through valve 154 and flow restrictor 155. Valve 152 may remain closed during the transfer process, or may be opened if additional oxygen enriched gas is needed. The selection of appropriate flow restrictors 151 and 155, coupled with controlled opening of valve 154 allows a controlled amount of oxygen enriched gas to be sent from canister 304 to 302. In one form, the controlled amount of oxygen enriched gas is an amount sufficient to purge canister 302 and minimize the loss of oxygen enriched gas through venting valve 132 of canister 302. While venting of canister 302 has been described, it should be understood that the same process can be used to vent canister 304 using flow restrictor 151, valve 152 and flow restrictor 153.

The pair of equalization/vent valves 152/154 work with flow restrictors 153 and 155 to optimize the air flow balance between the two canisters. This may allow for better flow control for venting the canisters with oxygen enriched gas from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched gas flowing from canister 304 toward canister 302 has a flow rate faster through valve 152 than the flow rate of oxygen enriched gas flowing from canister 302 toward canister 304 through valve 152. If a single valve was to be used, eventually either too much or too little oxygen enriched gas would be sent between the canisters and the canisters would, over time, begin to produce different amounts of oxygen enriched gas. Use of opposing valves and flow restrictors on parallel air pathways may equalize the flow pattern of the oxygen between the two canisters. Equalising the flow may allow for a steady amount of oxygen available to the patient over multiple cycles and also may allow a predictable volume of oxygen to purge the other of the canisters. In some forms, the air pathway may not have restrictors but may instead have a valve with a built in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may be adsorbed by the gas separation adsorbent. Adsorption of water inside the canisters may lead to gradual degradation of the gas separation adsorbents, steadily reducing ability of the gas separation adsorbents to produce oxygen enriched gas.

In one form, outside air may be inhibited from entering canisters after the oxygen concentrator is shut down by pressurising both canisters prior to shutdown. By storing the canisters under a positive pressure, the valves may be forced into a hermetically closed position by the internal pressure of the air in the canisters. In one form, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In one form, the pressure in the canisters, at shutdown, is at least greater than standard atmospheric pressure (i.e., greater than 760 mmHg (Torr), 1 atm, 101,325 Pa). In one form, the pressure in the canisters, at shutdown, is at least about 1.1 times greater than ambient pressure; is at least about 1.5 times greater than ambient pressure; or is at least about 2 times greater than ambient pressure.

In one form, pressurization of the canisters may be achieved by directing pressurized air into each canister from the compression system and closing all valves to trap the pressurized air in the canisters. In one form, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and or oxygen enriched gas from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternative form, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 7B, one form of an oxygen concentrator 100 is depicted. Oxygen concentrator 100 includes a compression system 200, a canister assembly 300 with air inlet 306, and a power supply 180 disposed within an outer housing 170. Outer housing 170 includes compression system inlets 105, cooling system passive inlet 101 at each end of outer housing 170, and outlet port 174. Inlets 101 are located in outer housing 170 to allow air from the environment to enter oxygen concentrator 100 to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator. Outlet port 174 is configured to attach to a conduit 192 (described below) to provide oxygen enriched gas produced by the oxygen concentrator 100 to a patient. Oxygen concentrator 100 may include a pressure sensor 176 coupled to controller 400 to determine an ambient pressure.

In one form, oxygen enriched gas produced in either of canisters 302 and 304 is collected in an oxygen accumulator 106 through check valves 142 and 144, respectively, as depicted schematically in FIG. 7A, before being provided to the patient.

### 5.7.1 Outlet System

FIG. 7C is a schematic diagram of an outlet system for an oxygen concentrator 100 according to one form of the present technology. A supply valve 160 may be situated within the gas flow path to control the release of the oxygen enriched gas from accumulator 106 to the patient. In one form, supply valve 160 is an electromagnetically actuated plunger valve. Supply valve 160 is actuated by controller 400 to control the delivery of oxygen enriched gas to a patient. Actuation of supply valve 160 is not timed or synchronized to the pressure swing adsorption process. Instead, actuation is, in POD therapy, synchronized to the patient's breathing, as described in more detail below. Additionally, supply valve 160 may have continuously-valued actuation to enable provision of oxygen enriched gas according to a predetermined flow rate profile.

Oxygen enriched gas in accumulator 106 passes through supply valve 160 into expansion chamber 162 as depicted in FIG. 7C. Oxygen enriched gas in expansion chamber 162 builds briefly, through release of gas from accumulator 106 by supply valve 160, and then is bled through a small orifice flow restrictor 175 to a flow rate sensor 185 and then to particulate filter 187. Flow restrictor 175 may be a 0.025 D flow restrictor. Other flow restrictor types and sizes may be used. In some forms, the diameter of the flow restrictor 175 may be variable by the controller 400 to allow the controller 400 to control the flow rate of delivered oxygen enriched gas. Flow rate sensor 185 may be any sensor capable of generating a signal representative of the flow rate of oxygen enriched gas flowing through the conduit. Particulate filter 187 may be used to filter bacteria, dust, granule particles, etc., prior to delivery of the oxygen enriched gas to the patient. The oxygen enriched gas passes through filter 187 to connector 190 which sends the oxygen enriched gas to the patient via outlet port 174 and to pressure sensor 194. In some embodiments, pressure sensor 194 may generate a signal that is proportional to the amount of positive or negative pressure applied to a sensing surface. The controller 400 may use the flow rate signal from the flow rate sensor 185 as a feedback signal to enable the closed-loop control of the continuously-valued actuation of the supply valve 160 in order to deliver a bolus of oxygen enriched gas according to a predetermined flow rate profile.

Expansion chamber 162 may include one or more oxygen sensors 165 capable of being used to determine an oxygen concentration of gas passing through the chamber. An oxygen sensor is a device capable of detecting oxygen in a gas. Examples of oxygen sensors include, but are not limited to, ultrasonic oxygen sensors, electrical oxygen sensors, and optical oxygen sensors. In one form, oxygen sensor 165 is an ultrasonic oxygen sensor that includes an ultrasonic emitter 166 and an ultrasonic receiver 168. In some forms, ultrasonic emitter 166 may include multiple ultrasonic emitters and ultrasonic receiver 168 may include multiple ultrasonic receivers. In forms having multiple emitters/receivers, the multiple ultrasonic emitters and multiple ultrasonic receivers may be axially aligned (e.g., transverse to the gas mixture flow path, which may be perpendicular to the axial alignment).

Flow rate sensor 185 may be used to determine the flow rate of oxygen enriched gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. Flow rate sensor 185 may be coupled to controller 400.

In some forms, oxygen sensor 165 and flow rate sensor 185 may provide a measurement of an actual amount of oxygen being provided. For example, flow rate sensor 185 may measure a volume of gas (based on flow rate) provided and ultrasonic sensor system 165 may measure the concentration of oxygen of the oxygen enriched gas provided. These two measurements together may be used by controller 400 to determine an approximation of the actual amount of oxygen provided to the patient.

Oxygen enriched gas passes through flow rate sensor 185 to filter 187. The filtered oxygen enriched gas passes through filter 187 to connector 190. Connector 190 may be a “Y” connector coupling the outlet of filter 187 to pressure sensor 194 and outlet port 174. Pressure sensor 194, which is coupled to controller 400, may be used to monitor the pressure of the oxygen enriched gas passing through outlet port 174 to the patient.

Oxygen enriched gas may be provided to a patient through an outlet conduit 192 connected to outlet port 174. In one form, conduit 192 may be a silicone tube. Conduit 192 may be coupled to a patient using a patient interface 196, as depicted in FIG. 7D. Patient interface 196 is positioned proximate to a patient's airway (e.g., proximate to the patient's mouth and/or nose) to allow delivery of the oxygen enriched gas to the patient while allowing the patient to breathe air from the surroundings. Patient interface 196 may be any device capable of providing the oxygen enriched gas to nasal cavities or oral cavities. Examples of patient interfaces include, but are not limited to: nasal masks, nasal pillows, nasal prongs, nasal cannulas, and mouthpieces. Patient interface 196 is depicted as a nasal cannula in FIG. 7D. One example of such a nasal cannula being worn by a patient 1000 is illustrated as 3800 in FIG. 3B.

### 5.7.2 Triggering Bolus Release

As mentioned above, in POD mode, oxygen enriched gas is provided to the patient in synchrony with the breathing cycle. In order to minimize the amount of oxygen enriched gas that is needed to be produced, or conversely to minimise the wastage of oxygen enriched gas delivered during exhalation, controller 400 may be configured to synchronise delivery of the oxygen enriched gas with the patient's inhalations. Reducing the amount of oxygen delivered may reduce the amount of air compression needed for oxygen concentrator 100 (and consequently may reduce the power demand from the compressors).

In POD mode, oxygen enriched gas produced by oxygen concentrator 100 is stored in an oxygen accumulator 106 and released by supply valve 160 to the patient as a pulse or “bolus” as the patient inhales. In some implementations, the bolus comprises a rectangular pulse whose flow rate profile is constant throughout its duration.

In one implementation, a sensor such as the pressure sensor 194 may be used to determine the onset of inhalation. Typically, at the onset of inhalation, the patient begins to draw air into their lungs through the nose. As the air is drawn in, a drop in pressure is generated at the patient end of the conduit 192, due, in part, to the venturi action of air being drawn across the end of the conduit 192. Controller 400 may analyse the pressure signal from the pressure sensor 194 to detect such a drop in pressure, indicating the onset of inhalation. Upon detection of the onset of inhalation, controller 400 opens supply valve 160 to release a bolus of oxygen enriched gas from the accumulator 106. This is referred to as “triggering” the bolus release. A positive change or rise in the pressure indicates an exhalation by the patient. In one form, when the controller 400 detects a positive pressure change in the pressure signal from the pressure sensor 194, supply valve 160 is closed until the next onset of inhalation. Alternatively, supply valve 160 may be closed after a predetermined interval known as the bolus duration.

### 5.8 Supplementary Oxygen Performance

Oxygen concentrator 100 may act as the oxygen source for respiratory therapy with supplementary oxygen. In various forms of the present technology, as illustrated in FIG. 4B, supplementary oxygen 4180 may be delivered or “entrained” from the oxygen source at an insertion point in the pneumatic path, such as within the RPT device 4000 upstream of the pneumatic block 4020, within the air circuit 4170, and/or within the patient interface 3000 or 3800. The first two of these options are referred to as distal coupling of supplementary oxygen (i.e. distal to the patient), while the third is referred to as proximal coupling (i.e. proximal to the patient). In all cases, the patient interface 196 specific to the POC is not needed, and the POC 100 is connected to the insertion point via the conduit 192.

As mentioned above, POCs operating in POD mode traditionally do not function efficiently when coupled to the airpath of RPT devices to deliver supplementary oxygen, for at least two reasons.

Firstly, the pressure within the RPT device's air circuit 4170 may confound the POC's triggering scheme (which as described above is typically based on sensing a drop in outlet pressure by the pressure sensor 194). One solution is to trigger the POC in synchrony with the triggering of the RPT device 4000, such as by the central controller 4230 of the RPT device 4000 communicating with the controller 400 of the POC 100. In an example of such implementations, the POC 100 acts as a local external device 4288, in communication with the RPT device 4000 via the local external communication network 4284. Such implementations typically require a modification of the configuration of the POC controller 400, via the instructions stored in memory 420, to actuate the supply valve 160 in response to a triggering signal received from the central controller 4230 of the RPT device 4000, rather than the controller 400 detecting a drop in pressure from the pressure sensor 194. However, such re-configuration of the POC controller 400 is not always convenient.

Secondly, even if synchronous triggering of the POC 100 is successfully achieved, such that the controller 400 actuates the supply valve 160 at the instant of onset of inhalation, the bolus may not be received in time to reach the alveoli due to the propagation delay or latency of the oxygen circuit. Oxygen delivery efficiency with respect to how much of the released oxygen is available in time for the patient's respiration may therefore still be sub-optimal.

Even when the POC is operating in continuous flow mode, varying device flow rates over the respiratory cycle may lead to oxygen accumulation within the air circuit 4170 and “POD-like” behaviour at the patient's airway. Consequently, oxygen delivery efficiency may be sub-optimal in this mode as well.

### 5.8.1 POC Trigger Module

FIG. 8 is a block diagram illustrating a trigger module 800 for a POC 100 working in conjunction with an RPT device 4000 to deliver supplementary oxygen according to one form of the present technology. The trigger module 800 is configured to be positioned between the conduit 192 and the insertion point of supplementary oxygen. The trigger module 800 comprises a housing 830 configured to be pneumatically connected to the conduit 192 from the POC 100, and an output conduit 840 protruding from the housing 830 and configured to be pneumatically connected to the insertion point of supplementary oxygen, whether distally or proximally to the patient interface 3000 or 3800. Within the housing 830 is a piston 810 actuated by a solenoid 820. The current within the solenoid 820 is supplied from a power source such as the power supply 4210 of the RPT device 4000.

The supply of current to the solenoid 820 may be controlled by the controller 4230 of the RPT device 4000. In such a configuration the trigger module 800 may be a local external device 4288 as illustrated in FIG. 4C, communicating with the RPT device 4000 via a local external communication network 4284 as described above. Alternatively, the trigger module 800 may be housed within the RPT device 4000. In such an implementation, the insertion point of supplementary oxygen 4180 may be close to the start of the air circuit 4170. It will be recognized that any other form of communication (e.g., wired or wireless) between the trigger module 800 and the controller 4230 of the RPT device 4000 may be implemented.

When a command is issued by the controller 4230 of the RPT device 4000 to the trigger module 800, which may be received by the power source of the trigger module 800, the trigger module 800 is activated so that current flows to energise the solenoid 820 to actuate the piston 810. As a result, the piston may move, such as to withdraw within the housing 830 as indicated by the arrow 850. The effect of the movement of the piston is to impart a sudden drop in pressure in the conduit 192. The command issued by the controller may be considered a “pseudo-trigger” command that pneumatically induces triggering of release of a bolus of the POC. In this regard, the piston 810 is configured such that, when the output conduit 840 is connected to the air circuit 4170, the drop in pressure resulting from the withdrawal of the piston 810 is sufficient to be detected by the pressure sensor 194 in the POC 100 and thereby implement a pneumatic intermediary for triggering release of a bolus. As such, the POC's typical triggering process responds to the mechanized pneumatic pseudo-trigger by activating its trigger signal for release of a bolus. The bolus may then pass through the pneumatic path of the trigger module 800 and the output conduit 840 on the way to the patient interface 3000 or 3800. At a predetermined interval after triggering the bolus, the controller 4230 may issue a “return” or reset command, or otherwise deactivate the pseudo-trigger, to trigger module 800 such as to the power source of the trigger module 800, causing the current to withdraw from the solenoid 820. The piston 810 is then urged back to its original, un-actuated position by a spring mechanism to be ready for the next actuation.

### 5.8.2 Improving Oxygen Performance

While the trigger module 800 allows the controller 4230 to control the instant of bolus release (also referred to as the oxygen trigger point) from a typical POC 100 having its usual configuration, there remains the problem of determining when to activate the oxygen pseudo-trigger in relation to the patient's actual onset of inhalation. As mentioned above, the propagation delay of the oxygen circuit can affect oxygen delivery efficiency. To address such issues, versions of the present technology may implement a predictive triggering process that attempts to compensate for the propagation delay. With such predictive triggering, the activation of the oxygen pseudo-trigger is set to the onset of inhalation minus some predetermined amount of time, referred to as the “advance”, so that the bolus is triggered before the onset of inhalation by the amount of the advance. Predictive triggering depends on the ability to predict when the next onset of inhalation will occur, which may be performed by the central controller 4230 as part of the respiratory phase determination algorithm as described above. In one implementation, the advance is set to the propagation delay, so that the bolus arrives at the entrance to the airway at the instant of onset of inhalation. In another implementation, the advance is set to the propagation delay less an intentional delay, so that the bolus arrives at the entrance to the airway at the intentional delay after the onset of inhalation. An accurate estimate of the propagation delay is clearly helpful to predictive triggering.

The propagation delay increases with the volume of the oxygen circuit, and is therefore greatest for a distal coupling of the supplementary oxygen. The propagation delay may also be affected by the treatment pressure of the RPT device (in a respiratory pressure therapy system) or the treatment flow rate of the flow therapy device (in a flow therapy system), the vent characteristics of any vents, and (in a respiratory pressure therapy system) the patient's breathing pattern (e.g. tidal volume, breathing rate). In general, it is difficult to compute an accurate estimate of the propagation delay from all these variables of a given respiratory therapy system/patient combination.

In one implementation, a calibration process may be carried out to measure the propagation delay for a given respiratory therapy system/patient combination. The advance may then be set based on the measured propagation delay as described above.

The calibration process may use dedicated sensors, or sensors already resident in the therapy system. Such a process may involve the release of a bolus from the POC. The process may the run a timer starting from the release so as to measure the amount of time of the bolus's propagation until a time when a sensor detects it along the pneumatic path of the system. Some examples of such a calibration process may include:
A (fast) oxygen sensor at the patient interface end of the air circuit 4170 (the delivered oxygen may be detected as an increase in the oxygen concentration of the air that is provided from the RPT device).
A (fast) humidity sensor at the patient interface end of the air circuit 4170 (when a humidifier 5000 is present in the system, the delivered oxygen will lower the humidity of the air from the RPT device).
A pressure or flow rate sensor at the patient interface end of the air circuit 4170 (to sense the arrival time of a flow pulse, step, or chirp produced by control of the pressure generator 4140). The arrival time may be used to infer the volume of the air circuit 4170, which in turn may be used to estimate the propagation delay if the insertion point is known.

As an alternative to directly measuring the propagation delay via calibration, a model of pipe transport of gas mixtures may be used to model the system/patient combination and thereby estimate the fraction of inspired oxygen or the oxygen delivery efficiency of the combination as a function of the bolus advance and other parameters of the combination.

### 5.8.2.1 Pipe Transport Model

FIG. 9A contains an illustration of a pipe transport model. A mixture of (J+1) different gases flows along the pipe 900 from left to right at a flow rate q(t) that in general varies with time t (and can turn negative if flow is retrograde). The pipe 900, of volume V, is notionally partitioned into N cells, e.g. 910, each of volume V/N. The N cells are indexed by the integer n, where n=1, . . . , N. The mixture of (J+1) gases in the cell n at time t is represented by a J-vector xₙ(t) of mole fractions (relative concentrations). (Only J fractions are necessary to represent a mixture of J+1 gases since the mole fractions must add to one). Without loss of generality, the mole fraction of oxygen may be set to the first component x₁ₙ(t) of the mole fraction vector xₙ(t). For example, atmospheric air may be represented as a mixture of three gases (so J=2), i.e. nitrogen (79%), oxygen (21%) and carbon dioxide (0.04%), giving a mole fraction vector (0.21, 0.0004). The mole fraction of nitrogen, if needed, is computable from the mole fraction vector as one minus the sum of the J components.

The network of cells is driven by a shift variable s(t). The shift variable s(t) is non-dimensional and represents the progress of the gas mixture through the pipe 900. For example, s=1 represents progress of one cell in the positive direction, i.e. from left to right. The shift variable s(t) is constrained to the limits:

−1<s(t)<1 (1)

The shift variable s(t) changes over a time step Δt as follows:

$$s ( t + Δ t ) = s ( t ) + \frac{N}{V} q ( t ) Δ t \qquad ( 2 )$$

subject to the constraint (1). That is, the shift variable s changes by the fraction of a cell that is occupied by the volume of gas passing a point during the time step.

If in a given timestep s(t+Δt)≥1, i.e. at least one cell's volume of gas has shifted along the pipe, the mole fraction xₙ(t) in cell n is replaced by the mole fraction in cell n−1 (a single-cell positive shift):

xₙ(t+Δt)=xₙ₋₁(t) (3)

Similarly, if s(t+Δt)≤−1, the mole fraction xₙ(t) in cell n is replaced by the mole fraction in cell n+1 (a single-cell negative shift).

If s(t) increases to or above 1 as a result of the update in equation (2), the single-cell positive shift update in equation (3) is applied first, and the value of s is restored within its limits (1) by subtracting one from s.

Likewise, if s(t) decreases to or below −1 as a result of the update in equation (2), a single-cell negative shift update is applied first, and the value of s is restored within its limits (1) by adding one to s.

The pipe 900 is assumed to be terminated at each end by a reservoir of fixed or relatively slowly varying mole fraction. For the leftmost cell (i.e. n=1), xₙ₋₁ is the mole fraction vector of the cell's adjacent terminating reservoir. Likewise for the rightmost cell (i.e. n=N), xₙ₊₁ is the mole fraction vector of the cell's adjacent terminating reservoir.

FIG. 9B illustrates how the model handles branching points, e.g. supplementary gas insertion points or venting points. FIG. 9B shows a branching point 950 comprising pipe A (whose last two cells are indexed by N−1 and N) abutting pipe B (whose first cell is indexed by 1) at the point C into which flow gas mixtures x_{A}(t), x_{B} (t) and x_{C} (t) at respective flow rates q_{A}, q_{B}, and q_{C} (the sum of which must be zero). Pipes A and B terminate at a notional reservoir of zero width whose mole fraction is x′(t).

If q_{C} is positive, at least one of q_{A} and q_{B} must be negative. If only q_{B} is negative, then the mole fraction x′(t) at the reservoir is an average of the mole fractions x_{A} and x_{C}, weighted according to the flow rates q_{A} and q_{C} (both of which are positive):

$${x}^{′} ( t ) = \frac{{q}_{A}}{{q}_{A} + {q}_{C}} {x}_{A} ( t ) + \frac{{q}_{C}}{{q}_{A} + {q}_{C}} {x}_{C} ( t ) \qquad ( 4 )$$

If both q_{A} and q_{B} are negative, then x′(t)=x_{C}(t).

If q_{C} is negative, at least one of q_{A} and q_{B} must be positive. If only q_{A} is positive, then x′(t)=x_{A}(t). If only q_{B} is positive, then x′(t)=x_{B} (t). If both q_{A} and q_{B} are positive, then x′(t) is an average of the mole fractions x_{A} and x_{B}, weighted according to the flow rates q_{A} and q_{B}:

$${x}^{′} ( t ) = \frac{{q}_{A}}{{q}_{A} + {q}_{B}} {x}_{A} ( t ) + \frac{{q}_{B}}{{q}_{A} + {q}_{B}} {x}_{B} ( t ) \qquad ( 5 )$$

In the case where the branching point 950 models an insertion point of supplementary gas between two pipes A and B, assuming that q_{A} is positive, the equations (2) to (3) are first applied to model the pipe transport in pipe A and compute x_{A} (t) as x_{N}(t). Equation (4) is then applied to compute x′(t) including the effect of the delivered supplementary gas at the insertion point, setting q_{C} to the supplementary gas flow rate Qsupp(t) (which is never negative) and x_{C} to the mole fraction of the supplementary gas. Equations (2) to (3) are then applied to model the pipe transport in pipe B, using x′(t) as the pipe's leftmost terminating mole fraction vector.

In the case where the branching point 950 models a vent between two pipes A and B, first determine whether the vent flow Qv is positive or negative. If Qv is negative, proceed as for an insertion point, setting q_{C} to the negative of the vent flow rate Qv(t) and x_{C} to the mole fraction of ambient air. If Qv is positive, and assuming that q_{A} is positive, the equations (2) to (3) are first applied to model the pipe transport in pipe A and compute x_{A}(t) as x_{N}(t). The flow rate q_{B} is computed as the vent flow rate Qv(t) minus the incoming flow rate q_{A}. If the flow rate q_{B} is positive, the equations (2) to (3) are first applied to model the pipe transport in pipe B and compute x_{B} (t) as x₁(t). Equation (5) is then applied to compute x′(t) including the effect of the vent. If the flow rate q_{B} is negative, then x′(t) is set to x_{A}(t) and Equations (2) to (3) are then applied to model the pipe transport in pipe B, using x′(t) as the pipe's leftmost terminating mole fraction vector.

### 5.8.2.2 Estimating Oxygen Performance Using the Pipe Transport Model

FIG. 10 illustrates how a respiratory therapy system with a single-limb air circuit and supplementary gas delivery may be modelled by the pipe transport model. The gas may be modelled as a four-gas mixture (nitrogen, oxygen, carbon dioxide, and water vapour), so J=3, with oxygen represented by the first component x₁ of the mole fraction vector x, and carbon dioxide by the second component x₂. The complete air circuit 1010 consists of three connected pipes:
a first pipe (1015) modelling the portion of the conduit 4170 upstream of the insertion point;
a second pipe (1020) modelling the portion of the conduit 4170 downstream of the insertion point, and
a third pipe (1030) modelling the patient's anatomical deadspace.

The direction of positive flow rate is from left to right. The left or device end of the first pipe 1015 is modelled as a reservoir 1040 of infinite volume with the mole fraction of humidified atmospheric air (e.g. x₍ₐₜₘ₎=(0.197, 0.000295, 0.0619)). The flow rate of air entering the pipe 1015 from the RPT device 1040 is the device flow rate Qd(t). Supplementary oxygen is delivered at a flow rate Qsupp(t) at the branching point between pipe 1015 and pipe 1020. In POD mode, the oxygen flow rate Qsupp(t) is a rectangular pulse that starts at the advance before the onset of inhalation and lasts for the bolus duration at a bolus flow rate of Qb, as illustrated in FIG. 11. The bolus volume is the product of Qb and the bolus duration. The mole fraction x_{C} in the mixing equation (4) is set to the mole fraction x₍ₛᵤₚₚ₎ of the delivered supplementary gas (e.g. for pure, dry oxygen, x₍ₛᵤₚₚ₎=(1, 0, 0)).

Gas vents from the branching point between pipes 1020 and 1030 at the vent flow rate Qv(t), which models the vent 3400 of the patient interface 3000 or 3800. The branching point between pipes 1020 and 1030 is also the entrance to the patient's airway. Gas traverses the anatomical deadspace pipe 1030, exits the pipe 1030, and enters the lungs at the respiratory flow rate Qr(t). The lungs may be modelled by a reservoir 1050 of infinite volume with the mole fraction of exhaled air (e.g. x₍ₑₓₕ₎=(0.13, 0.05, 0.062)). (A more sophisticated model of the lungs, i.e. a reservoir of finite volume with a dynamic model of gas exchange to determine the exhaled mole fraction vector as a function of time, may be used instead of a fixed mole fraction of exhaled air.) The pipe 1030 may be assigned a predetermined volume (e.g. 150 ml for an adult) to model the anatomical deadspace with acceptable accuracy. Alternatively, the pipe 1030 may be assigned a volume VDₐₙ of anatomical deadspace estimated from the patient's height. In one implementation, the anatomical deadspace volume VDₐₙ may be estimated from the patient's height H using the following formula:

VD_{D}=7.585×10⁻⁴×H (cm)^{2.363} (6)

The time step Δt used in the pipe transport model may be set to the sampling interval of the flow rate and pressure sensors 4274 and 4274, or some multiple thereof. The number N of cells in each pipe may be chosen as an acceptable compromise between spatial resolution of the travelling bolus and computational demand. However, the time step Δt imposes an upper bound on N, in that N and Δt should be chosen such that Δs (the change in the shift variable s in one time step Δt, using (2)) does not exceed 0.5, for the highest flow rate q(t) likely to be encountered.

At each time step, the pipe transport model of the anatomical deadspace pipe 1030 returns a mole fraction vector x_{N}(t) of the gas mixture at the entrance to the patient's lungs 1050. The first component x_{1N}(t) of this vector x_{N}(t) is an estimate of the fraction of inspired oxygen (FiO₂). The FiO₂ estimate, optionally averaged over the inspiratory portion, may be used as an oxygen performance metric, since the greater its value, the more supplementary oxygen the patient is receiving.

The FiO₂ estimate x_{1N}(t) may be used to estimate the volume of oxygen supplementation due to the respiratory therapy for each breath. In the absence of respiratory therapy, the “expected” volume of oxygen entering the lung during a single breath is equal to the sum of rebreathed oxygen and inspired atmospheric oxygen. The volume of rebreathed oxygen is equal to the product of the anatomical deadspace volume and the mole fraction of oxygen in exhaled air, while the volume of inspired atmospheric oxygen is equal to the product of the inspiratory volume Vᵢ less the deadspace volume VDₐₙ and the mole fraction of oxygen in atmospheric air:

V_{O}_{₂}_{(expected)}=VDₐₙ×x₁₍ₑₓₕ₎+(Vᵢ−VDₐₙ)×x₁₍ₐₜₘ₎ (7)

This is an anatomical quantity that is slightly less than 0.21 times the inspiratory volume Vᵢ.

The flow rate Qr(t) of oxygen entering the lung may be multiplied by the FiO₂ estimate and integrated over the inspiratory portion of the breathing cycle to obtain the volume of oxygen entering the lung in one breath.

$${V}_{{O}_{2} ( l u n g )} = {\int }_{insp}^{ } Qr ( t ) {x}_{1 N} ( t ) d t \qquad ( 8 )$$

The volume of oxygen supplementation due to the respiratory therapy during a single breath is equal to the volume of oxygen entering the lung minus the volume of oxygen expected to enter the lung in the absence of therapy:

V_{O}_{₂}₍ₛᵤₚₚ₎=V_{O}_{₂}_{(lung)}−V_{O}_{₂}_{(expected)} (9)

The volume of oxygen supplementation per breath V_{O}_{₂}₍ₛᵤₚₚ₎ may be used as an oxygen performance metric, since the greater its value, the more supplementary oxygen the patient is receiving.

The oxygen supplementation ratio is the ratio of the volume V_{O}_{₂}₍ₛᵤₚₚ₎ of oxygen supplementation per breath to the inspiratory volume Vᵢ. It may be shown that the oxygen supplementation ratio ranges between −0.21 (when the respiratory therapy amounts to breathing in a closed bottle, so the patient receives no oxygen at all in the long term) and 0.79 (when the patient receives 100% oxygen at the alveoli). The oxygen supplementation ratio may also be used as an oxygen performance metric, since the greater its value, the more supplementary oxygen the patient is receiving, normalised for the size of the patient's inspiratory volume.

The oxygen delivery efficiency is the volume V_{O}_{₂}₍ₛᵤₚₚ₎ of oxygen supplementation per breath divided by the bolus volume. The oxygen delivery efficiency may also be used as an oxygen performance metric, since the greater its value (which has a maximum of one), the more efficient is the delivery of supplementary oxygen, i.e. the less delivered oxygen is wasted.

The pipe transport model may be employed either wholly theoretically (“offline”) or partly empirically (“online”) to estimate the oxygen performance metrics. The difference between theoretical and empirical is whether the respiratory flow rate profile Qr(t) is known or unknown respectively. An empirical approach may be taken if the respiratory flow rate profile Qr(t) is unknown. Under the empirical approach, the therapy system is set up, therapy is commenced for the patient, and the device flow rate Qd(t) and device pressure Pd(t) are measured. For pressure therapies, the vent flow rate and respiratory flow rate profiles Qv(t) and Qr(t) may be estimated using the measured device flow rate Qd(t) via the pressure therapy algorithms described above based on the characteristics of the air circuit 4170 and the vent 3400. For flow therapies, the vent flow rate and respiratory flow rate profiles Qv(t) and Qr(t) may be estimated using the measured device flow rate Qd(t), the measured device pressure Pd(t), and the characteristics of the air circuit 4170 via the flow therapy algorithms described above. The pipe transport model, such as by applying (7), (8) and (9) with a controller or other processor, may then be employed to estimate the oxygen performance metrics as all the flow rates Qd(t), Qsupp(t), Qv(t), and Qr(t) in FIG. 10 are known.

The theoretical approach does not need any therapy to be applied, so the device flow rate Qd(t) is unknown. Instead, an approximation of the respiratory flow rate profile Qr(t) of the patient may be obtained by fitting a model flow rate profile, such as the profile of FIG. 6, to various breathing parameters of the patient (the tidal volume, the breathing rate, and the duty cycle). In one implementation, these parameters may be estimated from the patient's height. One method of estimating breathing parameters from a patient's height is disclosed in PCT Publication No. WO2019/036768, the entire content of which is incorporated herein by reference. For pressure therapies, the device flow rate Qd(t) may be estimated from the respiratory flow rate profile Qr(t), the pressure therapy parameters (i.e. the IPAP and the EPAP, which parametrise the treatment pressure profile Pt(t)), and the vent characteristic Qv(Pm), using the device flow rate estimation algorithm described above. The pipe transport model may then be employed to estimate the oxygen performance metrics as all the flow rates Qd(t), Qsupp(t), Qv(t), and Qr(t) in FIG. 10 are known.

For high flow therapy, the vent characteristic Qv(Pm) for an unsealed patient interface 3800 is unknown. However, the device flow rate Qd is known, as it is controlled to a treatment flow rate profile Qt(t) that is nominally greater at all times than the respiratory flow rate Qr. The vent flow rate Qv(t) is therefore always positive regardless of the respiratory flow rate Qr. An approximation to the respiratory flow rate profile Qr(t) of the patient may be obtained from the patient height as described above. The vent flow rate Qv(t) may then be calculated as

Qv(t)=Qd(t)+Qox(t)−Qr(t) (10)

The pipe transport model may then be employed to estimate the oxygen performance metrics as all the flow rates Qd(t), Qsupp(t), Qv(t), and Qr(t) in FIG. 10 are known.

FIG. 12 is a flow chart illustrating a method 1200 of estimating an oxygen performance metric according to one aspect of the present technology. The method 1200 may be implemented by one or more of the controllers described herein or other processing device(s) (processor based) described herein.

The method 1200 embodies the theoretical or offline approach rather than the empirical or online approach described above, in that no therapy data from device sensors is used to estimate the oxygen performance metric.

The method 1200 starts at step 1210, which estimates the respiratory flow rate profile Qr(t). In one implementation, the respiratory flow rate profile Qr(t) may be obtained by fitting a model flow rate profile, such as the profile of FIG. 6, to various breathing parameters of the patient (the tidal volume, the breathing rate, and the duty cycle). These parameters may be estimated from the patient's height 1250 as described above.

Step 1220 follows, at which the other system flow rates (the vent flow rate Qv(t), the device flow rate Qd(t), and the oxygen flow rate Qsupp(t)) are estimated or determined. The implementation of step 1220 varies depending on the type of therapy. For respiratory pressure therapy, the pressure therapy algorithms described above may be used to estimate the vent flow rate Qv(t) and the device flow rate Qd(t), using the pressure therapy parameters 1260 (the EPAP and the IPAP pressures, which determine the treatment pressure profile Pt(t)) and the air circuit parameters 1280 (the pressure drop characteristic ΔP(Q) and the vent characteristic Qv(Pm)), as well as the respiratory flow rate profile Qr(t) estimated in step 1210. The oxygen flow rate Qsupp(t) is determined from the supplementary oxygen parameters 1270 (the bolus flow rate Qb, the advance, and the bolus duration).

For flow therapy, step 1220 sets the device flow rate Qd(t) to be equal to the treatment flow rate profile Qt(t) (the therapy parameter 1260), determines the oxygen flow rate Qsupp(t) from the supplementary oxygen parameters 1270 (the bolus flow rate Qb, the advance, and the bolus duration) and estimates the vent flow rate Qv(t) using equation (10).

At the next step 1230, the pipe transport model is applied as described above to estimate the desired oxygen performance metric using the system flow rates estimated or determined at step 1220 and the circuit parameters 1280 (the total volume V, the anatomic deadspace volume VDₐₙ and the oxygen delivery cell n_{d}). The desired oxygen performance metric may be computed from the oxygen mole fraction x_{1N}(t) and the respiratory flow rate Qr(t) applying computing or programming functions that implement equations (6) to (9) as described above.

FIG. 13 is a flow chart illustrating a method 1300 of estimating an oxygen performance metric according to one aspect of the present technology. The method 1300 may be implemented by one or more of the controllers described herein or other processing device(s) (processor based) described herein.

The method 1300 embodies the empirical rather than the theoretical approach described above, in that therapy data from device sensors is used to estimate the oxygen performance metric “online” during respiratory pressure therapy.

The method 1300 starts at step 1310, which uses the sensor data 1350 (the measured device pressure Pd(t) and the measured device flow rate Qd(t)) to estimate the vent flow rate Qv(t) and the respiratory flow rate Qr(t) using the pressure therapy algorithms or the flow therapy algorithms described above. Step 1310 uses the circuit parameters 1370, namely the pressure drop characteristic ΔP(Q) of the air circuit 4170 and the characteristic Qv(Pm) of the vent 3400 (for pressure therapies). Step 1310 also determines the oxygen flow rate Qsupp(t) from the supplementary oxygen parameters 1360 (the bolus flow rate Qb, the advance, and the bolus duration).

At the next step 1320, the pipe transport model is applied as described above to estimate the desired oxygen performance metric using the system flow rates estimated or determined at step 1320 and the circuit parameters 1370 (the total volume V, the anatomic deadspace volume VDₐₙ and the oxygen delivery cell n_{d}). The desired oxygen performance metric may be computed from the oxygen mole fraction x_{1N}(t) and the respiratory flow rate Qr(t) by applying computing or programming functions that implement equations (6) to (9) as described above.

### 5.8.2.3 Improvement/Optimisation Methods

It may be seen that many parameters of the therapy system/patient combination influence the flow rates necessary for application of the pipe transport model to estimate oxygen performance metrics. These parameters may be categorised as potentially auto-controllable, i.e. controllable directly by the central controller 4230 and/or the controller 400, potentially manually controllable, or not controllable. The potentially auto-controllable parameters are:
Therapy parameters: IPAP, EPAP (bi-level respiratory pressure therapy), or treatment flow rate profile Qt(t) (flow therapy)
Supplementary oxygen parameters: bolus advance, duration, and bolus flow rate Qb

(In continuous flow mode, there is no discrete bolus. Instead the supplementary oxygen flow rate Qsupp(t) is constant at Qb throughout the breathing cycle. This means the advance and the bolus duration are not available as controllable parameters to improve the oxygen performance.)

The potentially manually controllable parameters are:
Air circuit parameters: length, diameter (which determine the volume Vas well as the pressure drop characteristic ΔP(Q))
Vent characteristic Qv(Pm), which depends on the type of patient interface
Oxygen insertion point

These potentially controllable parameters (whether auto- or manually controllable) may be continuously variable within respective ranges. Examples are:
the bolus advance or the EPAP (auto-controllable);
the length of the air circuit 4170, by concertina-style extension or contraction (manual).

Alternatively, the potentially controllable parameters may be variable between discrete alternatives. Examples are:
the bolus flow rate Qb may be auto-controllable between discrete values corresponding to different user settings of the POC;
the air circuit 4170 may be manually selectable between several conduits of differing length and diameter;
the vent characteristic may be manually selectable between the characteristics of the vents 3400 of several different types of patient interface 3000 or auto-controllable between various opening settings of a servo-mechanical version of vent 3400;
the oxygen insertion point may be manually selectable to be at the RPT device 4000 or at the patient interface 3000 or 3800 or at discrete locations in between.

The parameters that are not controllable are the patient characteristics such as breathing parameters (e.g. tidal volume, breathing rate, and duty cycle), which parametrise the respiratory flow rate profile Qr(t), and height.

In any given scenario, only some of the potentially controllable parameters may be practically controllable for the purposes of oxygen performance improvement. The remainder may be regarded as not controllable, being either set manually (e.g. the bolus flow rate Qb and duration), varying for other reasons (e.g. the IPAP and/or the EPAP), or the only one available (for discrete alternative parameters). These practically non-controllable parameter values, along with the genuinely non-controllable parameter values, may be provided to the controller 4230 of the RPT device 4000 in various ways:
by manual entry through the input device 4220;
via a “learn circuit” procedure;
by the controller 400 of the POC 100;
being under the direct control of the controller 4230.

Likewise, the non-controllable parameter values may be provided to the controller 400 of the POC 100 in various ways:
by manual entry through the control panel 600;
via a “learn circuit” procedure;
by the central controller 4230 of the RPT device 4000;
being under the direct control of the controller 400.

FIG. 14A is a flow chart illustrating a method 1400 of “pre-optimising” continuously controllable parameters of a therapy system/patient combination, i.e. determining a set of values of continuously controllable parameters that optimises an oxygen performance metric given the values of the remaining (discretely controllable and non-controllable) parameters. The method 1400 may be carried out offline, i.e. without any therapy data. The method 1400 may be implemented by one or more of the controllers described herein or other processing device(s).

The method 1400 starts at step 1410, which estimates the oxygen performance metric for the current values of the continuously controllable parameters given the values of the remaining parameters of the therapy system/patient combination. Step 1410 may be implemented using the “theoretical” method 1200 of FIG. 12. Step 1420 tests whether the oxygen performance metric estimated at step 1410 is satisfactory. In one implementation, step 1420 compares the oxygen performance metric estimated at the most recent iteration of step 1410 with a threshold such as a metric estimated at the preceding iteration of step 1410, and returns “Y” if the two values are similar (e.g., within a threshold indicating convergence). If step 1420 returns “Y”, the method 1400 concludes at step 1440, and the current values of the continuously controllable parameters are “optimal” for the given values of the remaining parameters of the therapy system/patient combination. Otherwise, if step 1420 returns “N”, step 1430 adjusts one or more of the values of the continuously controllable parameters so as to improve the oxygen performance metric given the values of the remaining parameters of the therapy system/patient combination. Step 1430 may be carried out using conventional multi-parameter optimisation methods such as gradient descent. The method 1400 then returns to step 1410.

FIG. 14B is a flow chart illustrating a method 1450 of “pre-optimising” the controllable parameters of a therapy system/patient combination, i.e. determining a set of values of controllable parameters (both discretely and continuously variable) that maximises an oxygen performance metric given the values of the remaining (non-controllable) parameters. Like the method 1400, the method 1450 may be carried out offline, i.e. without any therapy data. The method 1450 may be implemented by one or more of the controllers described herein or other processing device(s).

The method 1450 starts at step 1460, which chooses an initial combination of values for the discretely controllable parameters. Step 1470 follows, which optimises the continuously controllable parameters given the current values of the discretely controllable parameters and the values of the remaining, non-controllable parameters. Step 1470 may be implemented using the method 1400 of FIG. 14A. Step 1470 also records the optimal values of the continuously controllable parameters, the current combination of values for the discretely controllable parameters, and the optimal oxygen performance metric to which the optimal controllable parameters give rise, e.g. in a table. Step 1480 then determined whether all the possible combinations of discretely controllable parameters have been exhausted by successive iterations of step 1470. If not (“N”), step 1495 chooses the next combination of values for the discretely controllable parameters, and the method 1450 returns to step 1470. If so (“Y”), the method 1400 concludes at step 1490, which reviews all the optimal oxygen performance metrics stored at iterations of step 1470, and chooses the combination of values for the discretely controllable parameters which gave the highest optimal oxygen performance metric, along with the corresponding optimal values for the continuously controllable parameters. The result is the optimal controllable parameters of a therapy system/patient combination given the non-controllable parameter values.

FIG. 15 is a flow chart illustrating a method 1500 of “pre-optimising” the controllable parameters of a therapy system/patient combination. Like the methods 1400 and 1450, the method 1500 may be carried out offline, i.e. without any therapy data. The method 1500 may be implemented by one or more of the controllers described herein or other processing device(s). The method 1500 may be carried out once, before the start of therapy, or as often as the non-controllable parameters of the therapy system/patient combination are changed.

The method 1500 starts at step 1510, at which the values of the non-controllable parameters are obtained, for example through user entry via the interface of the RPT device 4000 or the POC 100, or by querying the settings of the RPT device 4000 and/or the POC 100. Step 1520 follows, which optimises the controllable parameters of the therapy system/patient combination, given the values of the non-controllable parameters obtained at step 1510 in relation to an oxygen performance metric of the therapy system/patient combination. Step 1520 may be implemented using the method 1450, for example. Step 1530 then recommends to the user the optimal values of the manually controllable parameters identified at step 1520, for example via the interface of the RPT device 4000 or the POC 100. The user may be prompted to confirm or disconfirm that each recommendation has been adopted. If adoption of a recommendation is disconfirmed, the corresponding manually controllable parameter may be reclassified as non-controllable and the method 1500 may return to step 1520 to re-execute the optimisation using the current value of the newly-classified non-controllable parameter. The final step 1540 adopts the optimal values of the auto-controllable parameters identified at step 1520 by setting the auto-controllable parameters to their respective optimal values.

FIG. 16 is a flow chart illustrating a method 1600 of improving an oxygen performance metric of a therapy system/patient combination during respiratory therapy with supplementary oxygen. The method 1600 may be carried out online as therapy data is available for analysis. The method 1600 may be implemented by one or more of the controllers described herein or other processing device(s). The method 1600 may be executed once through, or over repeated iterations during a therapy session, in order to adapt the auto-controllable parameters to any changes in non-controllable parameters of the therapy system/patient combination, e.g. a change in patient breathing patterns, or a change in respiratory therapy parameters.

The method 1600 starts at step 1610, which initialises the auto-controllable parameters of the therapy system/patient combination, and obtains values for the manually controllable and non-controllable parameters. Step 1620 delivers the respiratory therapy with supplementary oxygen with the current parameters. Step 1620 may be thought of as operating continuously throughout the execution of the method 1600 from this point onwards. During step 1620, sensor data (the measured device pressure Pd(t) and the measured device flow rate Qd(t)) is monitored and recorded. Step 1630 uses the recorded sensor data along with the current parameter values to estimate the oxygen performance metric of the therapy system/patient combination. Step 1630 may be implemented using the method 1300. Step 1640 follows, at which values of the auto-controllable parameters that would improve the oxygen performance metric given the values of the remaining parameters are computed. Step 1650 then adjusts the auto-controllable parameters to the improving values computed at step 1640. Optionally, the method 1600 may loop back to step 1630 to further adapt the auto-controllable parameters to any changes in non-controllable parameters of the therapy system/patient combination.

In an alternative implementation of the present technology, a target oxygen performance metric may be provided, such as an FiO₂ of 50%. The device, such as one or more of the controllers described herein or other processing device(s), then computes, using the pipe transport model, and recommends a combination of manually controllable parameters, and adopts values for auto-controllable parameters, that can achieve this target value for the patient's height and breathing patterns. As an example of the latter, the central controller 4230 may cause the display 4294 of the RPT device 4000 to display the message “To achieve this target, use a 15 mm tube and an X-series mask”, while setting the EPAP of a bi-level pressure therapy to 4 cmH₂O.

The above described methods 1200, 1300, 1400, 1450, 1500, 1600 may be carried out by the central controller 4230 of the RPT device 4000, the controller 400 of the POC 100, or both controllers operating in concert, passing the necessary data between them over the local external communication network 4284. Alternatively, the above described methods 1200, 1300, 1400, 1450, 1500, 1600 may be carried out by a remote external computing device 4286 such as a server, having been configured to receive the necessary data over the remote external communication network 4282. As previously described, such methodologies may be implemented with program instructions that may be stored on a suitable storage medium (e.g., memory), which when accessed and executed by a controller or processor describe herein, perform the operations of the methodologies.

### 5.9 Glossary

For the purposes of the present technology disclosure, in certain forms of the present technology, one or more of the following definitions may apply. In other forms of the present technology, alternative definitions may apply.

### 5.9.1. General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the therapy system or patient, and (ii) immediately surrounding the therapy system or patient.

For example, ambient pressure may be the pressure immediately surrounding or external to the body.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device, while the treatment flow rate, which represents a target value to be achieved by the device flow rate Qd, is given the symbol Qt. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Humidifier: A humidifying apparatus constructed and arranged, or configured with a physical structure to be capable of providing a therapeutically beneficial amount of water (H₂O) vapour to a flow of air to ameliorate a medical respiratory condition of a patient.

Leak: An unintended flow of air. In one example, leak may occur as the result of an incomplete seal between a mask and a patient's face. In another example leak may occur in a swivel elbow to the ambient.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal. In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt. The pressure in the pneumatic path proximal to an outlet of the pneumatic block (the device pressure) is given the symbol Pd.

Respiratory Pressure Therapy (RPT): The application of a supply of air to an entrance to the airways at a treatment pressure that is typically positive with respect to atmosphere.

Ventilator: A mechanical device that provides pressure support to a patient to perform some or all of the work of breathing.

### 5.9.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow rate profile.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

(inhalation) Time (Ti): The duration of the inspiratory portion of the respiratory flow rate profile.

(exhalation) Time (Te): The duration of the expiratory portion of the respiratory flow rate profile.

(total) Time (Ttot): The total duration between the start of one inspiratory portion of a respiratory flow rate profile and the start of the following inspiratory portion of the respiratory flow rate profile.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.9.3 Ventilation

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts, pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the inspiratory portion of the breathing cycle by the patient's efforts.

### 5.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.11 Reference Signs List

oxygen concentrator 100
inlets 101
inlets 105
muffler 108
accumulator 106
inlet valves 122
inlet valves 124
outlet 130
valve 132
muffler 133
outlet valves 134
check valve 142
check valve 144
flow restrictors 151
valve 152
flow restrictors 153
valve 154
flow restrictors 155
supply valve 160
expansion chamber 162
oxygen sensors 165
ultrasonic emitter 166
ultrasonic receiver 168
outer housing 170
fan 172
outlet port 174
small orifice flow restrictor 175
pressure sensor 176
power supply 180
flow rate sensor 185
filter 187
connector 190
conduit 192
pressure sensor 194
patient interface 196
compression system 200
canister assembly 300
canisters 302
canister 304
air inlet 306
controller 400
processor 410
memory 420
control panel 600
trigger module 800
piston 810
solenoid 820
housing 830
output conduit 840
arrow 850
pipe 900
branching point 950
patient 1000
pipe 1010
pipe portion 1030
reservoir 1040
reservoir 1050
method 1200
step 1210
step 1220
step 1230
therapy parameter 1260
supplementary oxygen parameters 1270
circuit parameters 1280
method 1300
step 1310
step 1320
sensor data 1350
supplementary oxygen parameters 1360
circuit parameters 1370
method 1400
step 1410
step 1420
step 1430
method 1450
step 1460
step 1470
step 1480
method 1500
step 1510
step 1520
step 1530
step 1540
method 1600
step 1610
step 1620
step 1630
step 1640
step 1650
patient interface 3000
seal-forming structure 3100
plenum chamber 3200
structure 3300
vents 3400
connection port 3600
forehead support 3700
unsealed patient interface 3800
RPT device 4000
external housing 4010
upper portion 4012
portion 4014
panel 4015
chassis 4016
handle 4018
pneumatic block 4020
air filter 4110
inlet air filter 4112
outlet air filter 4114
mufflers 4120
inlet muffler 4122
outlet muffler 4124
pressure generator 4140
blower 4142
motor 4144
anti-spill back valve 4160
air circuit 4170
supplementary oxygen 4180
electrical components 4200
PCBA 4202
power supply 4210
input devices 4220
central controller 4230
clock 4232
therapy device controller 4240
protection circuits 4250
memory 4260
transducers 4270
pressure sensors 4272
flow rate sensor 4274
motor speed transducer 4276
data communication interface 4280
remote external communication network 4282
local external communication network 4284
remote external device 4286
local external device 4288
output device 4290
display driver 4292
display 4294
therapy parameter determination
algorithm 4329
therapy control module 4330
humidifier 5000
humidifier inlet 5002
humidifier outlet 5004
humidifier base 50060
humidifier reservoir 5110
humidifier reservoir dock 5130
heating element 5240
prong 3810a
prong 3810b
lumen 3820a
lumen 3820b

1. A method of one or more processors for operation of apparatus configured to generate a respiratory therapy with supplementary oxygen for a respiratory disorder of a patient, the method comprising:
setting, by the one or more processors, one or more therapy parameters associated with delivering a flow of air to a patient interface via an air circuit of the apparatus;
setting, by the one or more processors, one or more supplementary oxygen parameters associated with inserting supplementary oxygen into the flow of air of the air circuit;
computing, by the one or more processors, an oxygen performance metric associated with (1) one or more characteristics of the patient and (2) the flow of air with the supplementary oxygen, the computing including applying one or more functions comprising the one or more therapy parameters and the one or more supplementary oxygen parameters; and
generating, by the one or more processors, output based on the computed oxygen performance metric.

2. The method of claim 1 wherein the output comprises a displayed indicator including the computed oxygen performance metric.

3. The method of any one of claims 1 to 2 wherein the output comprises an adjustment of one or more of:
the one or more therapy parameters; and
the one or more supplementary oxygen parameters,
for improving the oxygen performance metric.

4. The method of claim 3 wherein the adjustment comprises an automatic change to a setting of one or more controllers of the apparatus by the one or more processors.

5. The method of any one of claims 1 to 4, wherein the one or more functions comprises one or more parameters of the air circuit.

6. The method of any one of claims 1 to 5 wherein the one or more functions comprise a difference in a volume of oxygen entering the patient's lung and a volume of oxygen expected to enter the lung in an absence of therapy.

7. The method of claim 6 wherein the one or more functions comprise a ratio of the difference and an inspiratory volume of the patient.

8. The method of any one of claims 6 to 7 wherein the one or more functions comprise a ratio of the difference and a bolus volume of the supplementary oxygen.

9. The method of any of claims 1 to 5, wherein the oxygen performance metric is oxygen delivery efficiency.

10. The method of claim 9 wherein computing the oxygen performance metric comprises determining a ratio of (a) a difference in a volume of oxygen entering the patient's lung minus a volume of oxygen expected to enter the lung in an absence of therapy, and (b) a bolus volume of the supplementary oxygen.

11. The method of any one of claims 1 to 10 wherein the computing comprises applying a pipe transport model of the air circuit.

12. The method of claim 11 wherein the applying comprises generating a mole fraction vector of gas mixture at an entrance to the patient's lungs.

13. The method of any one of claims 1 to 12, wherein the computing the oxygen performance metric comprises accessing data comprising one or more signals representing measurement of properties of the flow of air using one or more sensors, the properties corresponding with either or both of (a) the one or more therapy parameters; and (b) the one or more supplementary oxygen parameters.

14. The method of claim 13 wherein
(a) the one or more therapy parameters comprise one or more of a device flow rate and a vent flow rate;
(b) the one or more supplementary oxygen parameters comprise a supplementary oxygen flow rate; and
(c) the one or more characteristics of the patient comprises a respiratory flow rate.

15. The method of any one of claims 1 to 12 wherein the computing comprises approximating a respiratory flow rate profile of the patient by fitting a model flow rate profile to breathing parameters of the patient.

16. The method of claim 15 wherein the breathing parameters comprise one or more of tidal volume, breathing rate, and duty cycle.

17. The method of any one of claims 1 to 16, wherein the respiratory therapy is respiratory pressure therapy, and the one or more therapy parameters comprise a treatment pressure.

18. The method of any one of claims 1 to 17, wherein inserting supplementary oxygen comprises operating an oxygen source in a pulsed oxygen delivery mode, wherein release of the supplementary oxygen is controlled with a bolus advance, a bolus duration, and a bolus flow rate.

19. The method of claim 18 further comprising generating a pseudo-trigger signal, the pseudo-trigger signal configured to activate a pneumatic intermediary module to generate a pressure drop for triggering of the release of the supplementary oxygen as a bolus.

20. The method of any one of claims 1 to 19, wherein the computing comprises:
estimating a vent flow rate of the patient using signals representing flow rate and pressure respectively of the flow of air and one or more parameters of the air circuit;
estimating a respiratory flow rate of the patient using the signals representing flow rate and pressure respectively of the flow of air; and
computing the oxygen performance metric using the vent flow rate, the respiratory flow rate, the signal representing flow rate, a supplementary oxygen flow rate, and one or more parameters of the air circuit.

21. The method of claim 20, wherein the one or more parameters of the air circuit comprise: an insertion point of the supplementary oxygen; and a volume of the air circuit.

22. A respiratory therapy system with supplementary oxygen, the system comprising:
a respiratory therapy device configured to generate a flow of air and adapted to pneumatically couple with (a) a patient interface configured to deliver the flow of air to an entrance to an airway of a patient, and (b) an air circuit configured to conduct the flow of air between the respiratory therapy device and the patient interface;
an oxygen source configured to insert supplementary oxygen into the flow of air; and
a controller configured to:
control setting of the respiratory therapy device to generate a respiratory therapy to the patient according to one or more therapy parameters;
control setting of the oxygen source to insert the supplementary oxygen into the flow of air according to one or more supplementary oxygen parameters;
compute an oxygen performance metric associated with (1) one or more characteristics of the patient and (2) the flow of air with the supplementary oxygen, the computing including applying one or more functions comprising the one or more therapy parameters and the one or more supplementary oxygen parameters; and
generate output based on the computed oxygen performance metric.

23. The respiratory therapy system of claim 22 wherein the output comprises a displayed indicator including the computed oxygen performance metric.

24. The respiratory therapy system of any one of claims 22 to 23 wherein the output comprises an adjustment of one or more of:
the one or more therapy parameters; and
the one or more supplementary oxygen parameters,
for improving the oxygen performance metric.

25. The respiratory therapy system of any one of claims 22 to 24, wherein the controller is a controller of the respiratory therapy device.

26. A respiratory therapy system for generating a respiratory therapy with supplementary oxygen for a respiratory disorder, the system comprising:
a respiratory therapy device configured to generate a flow of air and adapted to pneumatically couple with (a) a patient interface configured to deliver the flow of air to an entrance to an airway of a patient, and (b) an air circuit configured to conduct the flow of air between the respiratory therapy device and the patient interface;
an oxygen source configured to insert supplementary oxygen into the flow of air; and
a controller configured with processor executable instructions, the processor executable instructions configured to control operation of the system to generate the therapy, the processor executable instructions comprising instructions to perform the method of any one of claims 1 to 21.

27. A processor-readable medium, having stored thereon processor-executable instructions which, when executed by one or more processors of one or more controllers, cause the one or more controllers to control operation of apparatus configured to generate a therapy with supplementary oxygen for a respiratory disorder of a patient according to the method of any one of claims 1 to 21.

28. Apparatus comprising:
means for generating a flow of air;
means for delivering the flow of air to an entrance to an airway of a patient;
means for conducting the flow of air between the means for generating and the means for delivering;
means for inserting supplementary oxygen into the flow of air;
means for controlling the means for generating to deliver a respiratory therapy to the patient according to one or more therapy parameters;
means for controlling the means for delivering to insert the supplementary oxygen into the flow of air according to one or more supplementary oxygen parameters;
means for computing an oxygen performance metric associated with (1) one or more characteristics of the patient and (2) the flow of air with the supplementary oxygen, the computing including applying one or more functions comprising the one or more therapy parameters and the one or more supplementary oxygen parameters; and
means for generating output based on the computed oxygen performance metric.

29. A method of one or more processors for optimising one or more parameters of a respiratory therapy system with supplementary oxygen, the method of the one or more processors comprising:
optimising the one or more parameters of the respiratory therapy system with respect to an oxygen performance metric of the respiratory therapy system, to obtain optimal values of the one or more parameters;
setting automatically controllable ones of the one or more parameters of the respiratory therapy system to the respective optimal values of the automatically controllable ones of the one or more parameters; and
generating a recommendation to a user, via an interface of the respiratory therapy system, with the optimal values of manually controllable ones of the one or more parameters of the respiratory therapy system.

30. The method of claim 29, wherein the optimising comprises:
optimising, for each combination of discretely controllable ones of the one or more parameters, continuously controllable ones of the one or more parameters, giving the optimal values of the continuously controllable parameters for a current combination of discretely controllable parameters; and
selecting the combination of discretely controllable parameters for which the optimal values of the continuously controllable parameters give the highest oxygen performance metric, together with corresponding optimal values of the continuously controllable parameters.

31. The method of claim 30, wherein the optimising the continuously controllable parameters comprises, by the one or more processors:
estimating the oxygen performance metric for current values of the continuously controllable parameters;
adjusting values of the continuously controllable parameters so as to improve the oxygen performance metric; and
repeating the estimating and adjusting until the estimated oxygen performance metric satisfies a threshold.

32. The method of claim 31, wherein estimating the oxygen performance metric comprises:
estimating a respiratory flow rate of a patient based on a height of the patient;
estimating a vent flow rate of the respiratory therapy system using one or more parameters of the respiratory therapy and the estimated respiratory flow rate; and
estimating the oxygen performance metric using one or more parameters of an air circuit of the respiratory therapy system, the estimated vent flow rate, a respiratory therapy device flow rate, a supplementary oxygen flow rate, and the estimated respiratory flow rate.

33. A respiratory therapy system with supplementary oxygen, the system comprising:
a respiratory therapy device configured to generate a flow of air and adapted to pneumatically couple with (a) a patient interface configured to deliver the flow of air to an entrance to an airway of a patient, and (b) an air circuit configured to conduct the flow of air between the respiratory therapy device and the patient interface;
an oxygen concentrator configured to insert supplementary oxygen into the flow of air; and
a controller configured to:
optimise one or more parameters of the respiratory therapy system with respect to an oxygen performance metric of the respiratory therapy system, to obtain optimal values of the one or more parameters;
set automatically controllable ones of the one or more parameters of the respiratory therapy system to the respective optimal values of the automatically controllable ones of the one or more parameters; and
generate a recommendation to a user, via an interface of the respiratory therapy system, with optimal values of manually controllable ones of the one or more parameters of the respiratory therapy system.

34. Apparatus comprising:
means for generating a flow of air;
means for delivering the flow of air to an entrance to an airway of a patient;
means for conducting the flow of air between the means for generating and the means for delivering;
means for inserting supplementary oxygen into the flow of air;
means for optimising one or more parameters of the apparatus with respect to an oxygen performance metric of the apparatus, to obtain optimal values of the one or more parameters;
means for setting automatically controllable ones of the one or more parameters of the apparatus to the respective optimal values of the automatically controllable ones of the one or more parameters; and
means for generating a recommendation to a user with the optimal values of manually controllable ones of the one or more parameters of the apparatus.

35. A trigger module for a portable oxygen concentrator, the trigger module comprising:
a housing, the housing comprising an interior configured to be pneumatically connected to an outlet of the portable oxygen concentrator;
a piston within the housing configured to produce, when actuated, a drop in pressure within the interior; and
a solenoid configured, when energised, to actuate the piston.

36. The trigger module of claim 35, wherein the drop in pressure comprises a pneumatic pseudo-trigger capable of triggering the release of a bolus of oxygen from the portable oxygen concentrator when detected by a pneumatic sensor of the oxygen concentrator.

37. The trigger module of any one of claims 35 to 36, further comprising a spring mechanism configured to urge the piston toward its un-actuated position.

38. The trigger module of any one of claims 35 to 37 wherein the solenoid is configured to energise in response to a pseudo-trigger command generated by an external respiratory therapy device.

39. A method of one or more processors for computing an oxygen performance metric of a respiratory therapy system with supplementary oxygen, the method of the one or more processors comprising:
deriving an estimate of a respiratory flow rate of a patient from a height of the patient;
deriving an estimate of a vent flow rate of the respiratory therapy system with supplementary oxygen using one or more parameters of the respiratory therapy and the estimated respiratory flow rate; and
computing the oxygen performance metric using one or more parameters of an air circuit of the respiratory therapy system with supplementary oxygen, the estimated vent flow rate, a respiratory therapy device flow rate, a flow rate of the supplementary oxygen, and the estimated respiratory flow rate.

40. The method of claim 39, further comprising determining the supplementary oxygen flow rate using parameters of the supplementary oxygen.

41. The method of any one of claims 39 to 40, wherein the respiratory therapy is a respiratory pressure therapy, further comprising estimating the respiratory therapy device flow rate using the estimated respiratory flow rate and the one or more respiratory therapy parameters.

42. The method of any one of claims 39 to 40, wherein the respiratory therapy is a flow therapy, further comprising determining the respiratory therapy device flow rate using the one or more respiratory therapy parameters.

43. A respiratory therapy system with supplementary oxygen, the system comprising:
a respiratory therapy device configured to generate a flow of air according to one or more therapy parameters and adapted to pneumatically couple with (a) a patient interface configured to deliver the flow of air to an entrance to an airway of a patient, (b) an air circuit configured to conduct the flow of air between the respiratory therapy device and the patient interface, and (c) an oxygen source configured to insert supplementary oxygen into the flow of air; and
a controller configured to:
derive an estimate of a respiratory flow rate of the patient from a height of the patient;
derive an estimate a vent flow rate of the respiratory therapy system with supplementary oxygen using the one or more therapy parameters and the estimated respiratory flow rate; and
compute an oxygen performance metric of the respiratory therapy system with supplementary oxygen using one or more parameters of the air circuit, the estimated vent flow rate, a flow rate of the generated flow of air, a flow rate of the inserted supplementary oxygen, and the estimated respiratory flow rate.

44. Apparatus comprising:
means for generating a flow of air according to one or more therapy parameters;
means for delivering the flow of air to an entrance to an airway of a patient;
means for conducting the flow of air between the means for generating and the means for delivering;
means for inserting supplementary oxygen into the flow of air;
means for deriving an estimate of a respiratory flow rate of the patient from a height of the patient;
means for deriving an estimate of a vent flow rate of the apparatus using the one or more therapy parameters and the estimated respiratory flow rate; and
means for computing an oxygen performance metric of the apparatus using one or more parameters of the means for conducting, the estimated vent flow rate, a flow rate of the generated flow of air, a flow rate of the inserted supplementary oxygen, and the estimated respiratory flow rate.
